(19) United States
(12) Patent Application Publication
(10) Pub. No.: US 2022/0409675 A1
(43) Pub. Date: Dec. 29, 2022

(54) COMPOSITIONS COMPRISING BACTERIAL SPECIES AND METHODS RELATED THERETO

(71) Applicant: XBIOME INC., New York, NY (US)

(72) Inventors: Jason HUDAK, New York, NY (US); Jun MA, New York, NY (US); Jackie PAPKOFF, New York, NY (US)

(73) Assignee: XBIOME INC., New York, NY (US)

(21) Appl. No.: 17/756,274

(22) PCT Filed: Nov. 20, 2020
(86) PCT No.: PCT/US20/61586

§ 371 Date: May 20, 2022

Related U.S. Application Data
(60) Provisional application No. 62/939,120, filed on Nov. 22, 2019.

(51) Int. Cl. A61K 35/742; C12N 1/20; A61K 9/107; A61K 9/14

(52) U.S. Cl. CPC A61K 35/742; C12N 1/205; A61K 9/107; A61K 9/14

(57) ABSTRACT

The disclosure relates generally to bacterial strains of the genus Anaerostipes, e.g., Anaerostipes rhamnosivorans bacterial strains, and compositions, e.g., pharmaceutical compositions, comprising such bacterial strains. The disclosure further relates to method of using such bacterial strains and compositions for preventing or treating a disorder, e.g., an inflammatory disorder, a gastrointestinal disorder, a metabolic disorder, and/or dysbiosis when administered to a subject in need thereof.

### CROSS REFERENCE TO RELATED APPLICATIONS

This application claims the benefit of, and priority to, U.S. provisional patent application Ser. No. 62/939,120, filed on Nov. 22, 2019, which is hereby incorporated by reference herein in its entirety.

### SEQUENCE LISTING

The instant application contains a Sequence Listing which has been filed electronically in ASCII format and is hereby incorporated by reference in its entirety. Said ASCII copy, created on Nov. 20, 2020, is named ASP-060WO SL.txt and is 4,684,004 bytes in size.

### BACKGROUND

The gastrointestinal tract (GI), as well as other organ systems, is a complex biological system that includes a community of many different organisms, including diverse strains of bacteria. Hundreds of different species may form a commensal community in the gastrointestinal tract and other organs in a healthy person. Moreover, microorganisms present in the gut not only play a crucial role in digestive health, but also influence the immune system. A disturbance or imbalance in a biological system, e.g., the gastrointestinal tract, may include changes in the types and numbers of bacteria in the gut which may lead to the development of, or may be an indicator of, an unhealthy state and/or disease.

### SUMMARY

The disclosure relates generally to bacterial strains, bacterial strain mixtures, and compositions, e.g., comprising strains of the genus Anaerostipes. Disclosed bacterial strains, bacterial strain mixtures, or compositions are useful for treating gastrointestinal disorders and/or inflammatory disorders, including, for example, dysbiosis and/or immune mediated inflammatory disorders, such as inflammatory skin disorders. Exemplary inflammatory skin disorders include, but are not limited to, disorders associated with cell proliferation, such as psoriasis, eczema, dermatitis (e.g., eczematous dermatitides, topic and seborrheic dermatitis, allergic or irritant contact dermatitis, eczema craquele, photoallergic dermatitis, phototoxicdermatitis, phytophotodermatitis, radiation dermatitis, and stasis dermatitis), and acne.

In one aspect, provided herein is a composition, for example, a pharmaceutical composition, comprising a bacterial strain of the genus Anaerostipes. In some embodiments, the bacterial strain comprises a 16s rRNA gene sequence with at least about 97% sequence identity to the polynucleotide sequence of SEQ ID NO: 1. In a particular embodiment, the Anaerostipes bacterial strain is a strain of the species Anaerostipes rhamnosivorans. In some embodiments, the composition further comprises an excipient, diluent, and/or carrier. In some embodiments, the bacterial strain in the composition is lyophilized, freeze dried, or spray dried.

In some embodiments, the composition is capable of increasing production of at least one anti-inflammatory gene product, e.g., IL-10 and/or CCL-18, in a human cell, e.g., a macrophage (e.g., a THP-1 macrophage), a monocyte, a peripheral blood mononuclear cell (PBMC), or a monocyte-derived dendritic cell. For example, in some embodiments, the composition increases production of at least one anti-inflammatory gene product, e.g., IL-10 and/or CCL-18, in a human cell, e.g., a macrophage (e.g., a THP-1 macrophage), a monocyte, a peripheral blood mononuclear cell (PBMC), or a monocyte-derived dendritic cell, when the human cell is contacted with the composition. In some embodiments, the composition is capable of reducing or preventing disruption of, or increasing, barrier integrity of a human cell (e.g., an epithelial cell) monolayer, e.g., a HT29MTX-E12 cell monolayer treated with TNF-α. For example, in some embodiments, the composition reduces or prevents disruption of, or increases, barrier integrity of a human cell (e.g., an epithelial cell) monolayer, e.g., a HT29MTX-E12 cell monolayer treated with TNF-α, when the human cell monolayer is contacted with the composition.

In some embodiments, the composition comprises a bacterial strain of Anaerostipes that comprises a 16s rRNA gene sequence with at least about 98% sequence identity to the polynucleotide sequence of SEQ ID NO: 1. In some embodiments, the Anaerostipes bacterial strain comprises a 16s rRNA gene sequence with at least about 98.5%, 99%, or 99.5% sequence identity to the polynucleotide sequence of SEQ ID NO: 1. In some embodiments, the Anaerostipes bacterial strain comprises a 16s rRNA gene sequence of SEQ ID NO: 1. In some embodiments, the Anaerostipes bacterial strain shares at least 70% DNA-DNA hybridization with strain Anaerostipes sp. P127-B2a, deposited under accession number DSM 33275. In some embodiments, the Anaerostipes bacterial strain comprises a nucleotide sequence having at least about 70% identity to any one of SEQ ID NOs: 2-52. In some embodiments, the Anaerostipes bacterial strain comprises a genome having at least 95% average nucleotide identity (ANI) with the genome of Anaerostipes sp. strain P127-B2a, deposited under accession number DSM 33275. In some embodiments, the Anaerostipes bacterial strain comprises a genome having at least 96.5% average nucleotide identity (ANI) and at least 60% alignment fraction (AF) with the genome of Anaerostipes sp. strain P127-B2a, deposited under accession number DSM 33275. In some embodiments, the Anaerostipes bacterial strain is Anaerostipes sp. P127-B2a, deposited under accession number DSM 33275.

In some embodiments, the Anaerostipes bacterial strain of the composition is viable. In some embodiments, the bacterial strain is capable of at least partially colonizing an intestine of a human subject.

In some embodiments, the composition is suitable for oral delivery to a subject. In some embodiments, the composition is formulated as an enteric formulation. In some embodiments, the enteric formulation is formulated as a capsule, tablet, caplet, pill, troche, lozenge, powder, or granule. In some embodiments, the composition is formulated as a suppository, suspension, emulsion, or gel. In some embodiments, the composition comprises at least 1×10³ CFU of the bacterial strain.

In some embodiments, the composition comprises a therapeutically effective amount of the bacterial strain sufficient to prevent or treat a disorder when administered to a subject in need thereof. In some embodiments, the disorder is selected from the group consisting of an inflammatory disorder, a gastrointestinal disorder, inflammatory bowel disease, cancer, non-alcoholic fatty liver disease (NAFLD), non-alcoholic steatohepatitis (NASH), metabolic syndrome, insulin deficiency, insulin resistance-related disorders, insulin sensitivity, glucose intolerance, pre-diabetes, diabetes, high body mass index (BMI), excess adiposity, obesity, excess weight, cardiovascular disease, atherosclerosis, hyperlipidemia, hyperglycemia, abnormal lipid metabolism, and hypertension. In some embodiments, the gastrointestinal disorder is selected from the group consisting of ulcerative colitis, Crohn's disease, and irritable bowel syndrome. In some embodiments, the inflammatory disorder is an inflammatory skin disorder, for example, an inflammatory skin disorder selected from the group consisting of psoriasis, eczema, dermatitis (e.g., eczematous dermatitides, topic and seborrheic dermatitis, allergic or irritant contact dermatitis, eczema craquele, photoallergic dermatitis, phototoxicdermatitis, phytophotodermatitis, radiation dermatitis, and stasis dermatitis), and acne.

In some embodiments, the composition comprises an excipient selected from the group consisting of a filler, a binder, a disintegrant, and any combination(s) thereof In some embodiments, the excipient is selected from the group consisting of cellulose, polyvinyl pyrrolidone, silicon dioxide, stearyl fumarate or a pharmaceutically acceptable salt thereof, and any combination(s) thereof. In some embodiments, the composition further comprises a cryoprotectant. In some embodiments, the cryoprotectant is selected from the group consisting of a fructooligosaccharide, trehalose and a combination thereof. In some embodiments, the fructooligosaccharide is Raftilose® (fructooligosaccharide derived from inulin). In some embodiments, the composition is suitable for bolus administration or bolus release. In some embodiments, the composition comprises the Anaerostipes bacterial strain and at least one more additional bacterial strain(s).

In another aspect, provided herein is a bacterial strain, e.g., an isolated bacterial strain, of the genus Anaerostipes, wherein the bacterial strain comprises a 16s rRNA gene sequence with at least about 98% sequence identity to the polynucleotide sequence of SEQ ID NO: 1. In some embodiments, the Anaerostipes bacterial strain is capable of increasing production of at least one anti-inflammatory gene product, e.g., IL-10 and/or CCL-18, in a human cell, e.g., a macrophage (e.g., a THP-1 macrophage), a monocyte, a peripheral blood mononuclear cell (PBMC), or a monocyte-derived dendritic cell. For example, in some embodiments, the Anaerostipes bacterial strain increases production of at least one anti-inflammatory gene product, e.g., IL-10 and/or CCL-18, in a human cell, e.g., a macrophage (e.g., a THP-1 macrophage), a monocyte, a peripheral blood mononuclear cell (PBMC), or a monocyte-derived dendritic cell, when the human cell is contacted with the Anaerostipes bacterial strain. In some embodiments, the Anaerostipes bacterial strain is capable of reducing or preventing disruption of, or increasing, barrier integrity of a human cell (e.g., an epithelial cell) monolayer, e.g., a HT29MTX-E12 cell monolayer treated with TNF-α. For example, in some embodiments, the Anaerostipes bacterial strain reduces or prevents disruption of, or increases, barrier integrity of a human cell (e.g., an epithelial cell) monolayer, e.g., a

HT29MTX-E12 cell monolayer treated with TNF-α, when the human cell monolayer is contacted with the Anaerostipes bacterial strain.

In some embodiments, the Anaerostipes bacterial strain comprises a 16s rRNA gene sequence with at least about 98.5%, 99%, or 99.5% sequence identity to the polynucleotide sequence of SEQ ID NO: 1. In some embodiments, the Anaerostipes bacterial strain comprises a 16s rRNA gene sequence of SEQ ID NO: 1. In some embodiments, the Anaerostipes bacterial strain shares at least 70% DNA-DNA hybridization with strain Anaerostipes sp. P127-B2a, deposited under accession number DSM 33275. In some embodiments, the Anaerostipes bacterial strain comprises a nucleotide sequence having at least about 70% identity to any one of SEQ ID NOs: 2-52. In some embodiments, the Anaerostipes bacterial strain comprises a genome having at least 95% average nucleotide identity (ANI) with the genome of Anaerostipes sp. strain P127-B2a, deposited under accession number DSM 33275. In some embodiments, the Anaerostipes bacterial strain comprises a genome having at least 96.5% average nucleotide identity (ANI) and at least 60% alignment fraction (AF) with the genome of Anaerostipes sp. strain P127-B2a, deposited under accession number DSM 33275. In some embodiments, the Anaerostipes bacterial strain is Anaerostipes sp. P127-B2a, deposited under accession number DSM 33275. In some embodiments, the bacterial strain is capable of at least partially colonizing an intestine of a human subject.

In another aspect, provided herein is a food product comprising an Anaerostipes bacterial strain described herein, or a composition comprising an Anaerostipes bacterial strain described herein.

In another aspect, provided herein is a method of preventing or treating a disorder, for example, an inflammatory disorder, a gastrointestinal disorder, inflammatory bowel disease, cancer, non-alcoholic fatty liver disease (NAFLD), non-alcoholic steatohepatitis (NASH), metabolic syndrome, insulin deficiency, insulin resistance-related disorders, insulin sensitivity, glucose intolerance, pre-diabetes, diabetes, high body mass index (BMI), excess adiposity, obesity, excess weight, cardiovascular disease, atherosclerosis, hyperlipidemia, hyperglycemia, abnormal lipid metabolism, and hypertension in a subject in need thereof, the method comprising administering an Anaerostipes bacterial strain described herein or a composition comprising an Anaerostipes bacterial strain described herein (e.g., a therapeutically effective amount of an Anaerostipes bacterial strain described herein or a composition comprising an Anaerostipes bacterial strain described herein) to the subject. In some embodiments, the gastrointestinal disorder is ulcerative colitis, Crohn's disease or irritable bowel syndrome. In some embodiments the inflammatory disorder is an inflammatory skin disorder, for example, an inflammatory skin disorder selected from the group consisting of psoriasis, eczema, dermatitis (e.g., eczematous dermatitides, topic and seborrheic dermatitis, allergic or irritant contact dermatitis, eczema craquele, photoallergic dermatitis, phototoxicdermatitis, phytophotodermatitis, radiation dermatitis, and stasis dermatitis), and acne. Also provided herein is a method of treating a dysbiosis in a subject in need thereof, the method comprising administering an Anaerostipes bacterial strain described herein or a composition comprising an Anaerostipes bacterial strain described herein (e.g., a therapeutically effective amount of an Anaerostipes bacterial strain described herein or a composition comprising an Anaerostipes bacterial strain described herein) to the subject. Also provided herein is a method of modifying a gut microbiome in a subject, the method comprising administering an Anaerostipes bacterial strain described herein or a composition comprising an Anaerostipes bacterial strain described herein (e.g., a therapeutically effective amount of an Anaerostipes bacterial strain described herein or a composition comprising an Anaerostipes bacterial strain described herein) to the subject. In some embodiments of the methods provided herein, the method further comprises administering a prebiotic to the subject. In some embodiments, the subject is selected from the group consisting of a human, a companion animal, or a livestock animal.

### DESCRIPTION OF THE DRAWINGS

The disclosure can be more completely understood with reference to the following drawings.

FIG. 1 depicts a distance tree based on pairwise comparisons of the closest neighbors of Anaerostipes rhamnosivorans P127-B2a.

FIG. 2 depicts (A) a light micrograph and (B) a scanning electron micrograph of Anaerostipes rhamnosivorans P127-B2a bacterial cells (9.48k x magnification).

FIG. 3 depicts the short-chain fatty acid (SCFA) production profile of Anaerostipes rhamnosivorans P127-B2a cultured for 72 hours. Levels of butyric acid, propionic acid, and acetic acid in batch culture supernatants were analyzed by HPLC (ABPDU Berkeley CA).

Non-inoculated YCFAC media was used as a negative control.

FIG. 4 depicts the effect of Anaerostipes rhamnosivorans P127-B2a on HT29-MTX-E12 barrier integrity. The % change in TEER (ohms x cm²) between time 0 and 24 hour following basal compartment addition of TNF-α was plotted for 10% v/v of freshly cultured Anaerostipes rhamnosivorans P127-B2a (2 doses) and an unrelated bacterial strain negative control with no protective effect. The PBS control was set as baseline. Each test article was evaluated in 6 replicates and results are representative of at least two independent experiments. *p value ≤0.05 student t-test.

FIG. 5 depicts the effect of Anaerostipes rhamnosivorans P127-B2a on (A) CCL-18 and (B) IL-10 production in human THP-1 macrophages. THP-1 macrophages were co-cultured with PBS only or the Anaerostipes rhamnosivorans P127-B2a strain (3 doses), and supernatants were collected and assayed for production of the indicated cytokine. Each test article was evaluated in 4 replicates and results are representative of at least two independent experiments. p value ≤0.05 (one-way ANOVA).

FIG. 6 depicts the effect of Anaerostipes rhamnosivorans P127-B2a on (A) TNF-α, (B) IL-113, and (C) IL-12p40 production in human THP-1 macrophages. THP-1 macrophages were co-cultured with PBS only or the Anaerostipes rhamnosivorans P127-B2a strain (3 doses), and supernatants were collected and assayed for production of the indicated cytokine. Each test article was evaluated in 4 replicates and results are representative of at least two independent experiments. p value <0.05 (one-way ANOVA).

FIG. 7 depicts the effect of Anaerostipes rhamnosivorans P127-B2a on (A) TNF-α, (B) IL-1β, (C) TRAIL and (D) IFN-γ production in human peripheral blood mononuclear cells (PBMCs). PBMCs were co-cultured with PBS only or the Anaerostipes rhamnosivorans P127-B2a strain (2 doses), and supernatants were collected and assayed for production of the indicated cytokine. Each test article was evaluated in 4 replicates and results are representative of at least two independent experiments. p value <0.05 (one-way ANOVA).

FIG. 8 depicts the effect of Anaerostipes rhamnosivorans P127-B2a on (A) IL-17A, (B) IL-6, (C) IL-2 and (D) IL-10 production in human PBMCs. PBMCs were co-cultured with PBS only or the Anaerostipes rhamnosivorans P127-B2a strain (2 doses), and supernatants were collected and assayed for production of the indicated cytokine. Each test article was evaluated in 4 replicates and results are representative of at least two independent experiments. p value <0.05 (one-way ANOVA).

FIG. 9 depicts the effect of Anaerostipes rhamnosivorans P127-B2a on (A) TNF-α, (B) IL-1β, (C) TRAIL and (D) IFN-β production in human monocyte-derived dendritic cells (moDCs). moDCs were co-cultured with PBS only or the Anaerostipes rhamnosivorans P127-B2a strain (2 doses), and supernatants were collected and assayed for production of the indicated cytokine. Each test article was evaluated in 4 replicates and results are representative of at least two independent experiments. p value <0.05 (one-way ANOVA).

FIG. 10 depicts the effect of Anaerostipes rhamnosivorans P127-B2a on (A) IL-12p′70, (B) IL-6, (C) IL-23 and (D) IL-27 production in human moDCs. moDCs were co-cultured with PBS only or the Anaerostipes rhamnosivorans P127-B2a strain (2 doses), and supernatants were collected and assayed for production of the indicated cytokine. Each test article was evaluated in 4 replicates and results are representative of at least two independent experiments. p value <0.05 (one-way ANOVA).

FIG. 11 depicts the effect of Anaerostipes rhamnosivorans P127-B2a on IL-10 production in human moDCs. moDCs were co-cultured with PBS only or the Anaerostipes rhamnosivorans P127-B2a strain (2 doses), and supernatants were collected and assayed for production of IL-10. Each test article was evaluated in 4 replicates and results are representative of at least two independent experiments. p value <0.05 (one-way ANOVA).

FIG. 12 depicts the effect of oral dosing of Anaerostipes rhamnosivorans P127-B2a on back skin thickness in an Imiquimod-(IMQ) induced psoriasis-like skin inflammation mouse model. *p value<0.05, **p value<0.01, ***p value<0.005, and ****p value<0.001 (one-way ANOVA or two ANOVA with Fisher's LSD post test).

FIG. 13 depicts the effect of oral dosing of Anaerostipes rhamnosivorans P127-B2a on skin redness (erythema) in an oxazolone-induced atopic dermatitis mouse model. *p value<0.05, **p value<0.01, ***p value<0.005, and ****p value<0.001 (one-way ANOVA or two ANOVA with Fisher's LSD post test).

FIG. 14 depicts the effect of oral dosing of Anaerostipes rhamnosivorans P127-B2a on back skin scaling in an oxazolone-induced atopic dermatitis mouse model. *p value <0.05, **p value<0.01, ***p value<0.005, and ****p value<0.001 (one-way ANOVA or two ANOVA with Fisher's LSD post test).

FIG. 15 depicts the effect of oral dosing of Anaerostipes rhamnosivorans P127-B2a (AR) on (A) back skin thickness, and (B) total psoriasis area and severity index (PAST) score in an Imiquimod-(IMQ) induced psoriasis-like skin inflammation mouse model. *p value<0.05, **p value<0.01, ***p value<0.005, and ****p value<0.001 (one-way ANOVA or two ANOVA with Fisher's LSD post test).

FIG. 16 depicts the effect of oral dosing of Anaerostipes rhamnosivorans P127-B2a (AR) on skin levels of (A) IL-17A, (B) TNF-α, (C) IL-1β, (D) KC/GRO, and (E) IL-4 in an Imiquimod-(IMQ) induced psoriasis-like skin inflammation mouse model. *p value<0.05, **p value<0.01, ***p value<0.005, and ****p value<0.001 (one-way ANOVA or two ANOVA with Fisher's LSD post test).

FIG. 17 depicts the effect of oral dosing of Anaerostipes rhamnosivorans P127-B2a (AR) on (A) back skin thickness, and (B) total PASI score in a DNFB-induced atopic dermatitis mouse model. *p value<0.05, **p value<0.01, ***p value<0.005, and **** p value<0.001 (one-way ANOVA or two ANOVA with Fisher's LSD post test).

FIG. 18 depicts the effect of oral dosing of Christensenella sp. P152-H6d (ChA), and the combination of Christensenella sp. P152-H6d plus Faecalibacterium prausnitzii P162-C10a plus Anaerostipes rhamnosivorans P127-B2a (ChA +Fp +AR), respectively, on (A) skin redness (erythema), and (B) back skin thickness (edema) in a DNFB-induced atopic dermatitis mouse model. *p value<0.05, **p value<0.01, ***p value<0.005, and **** p value<0.001 (one-way ANOVA or two ANOVA with Fisher's LSD post test).

### DETAILED DESCRIPTION

### I. Bacterial Strains

A contemplated bacterial strain, for example, for use in a bacterial strain mixture, pharmaceutical composition or unit, or method provided herein, includes an Anaerostipes species strain. Exemplary Anaerostipes species include Anaerostipes butyraticus, Anaerostipes caccae, Anaerostipes hadrus, and Anaerostipes rhamnosivorans. Those of skill in the art will recognize that the genus Anaerostipes may undergo taxonomical reorganization. Thus, it is intended that a contemplated Anaerostipes species include Anaerostipes species that have been renamed and/or reclassified, as well as those that may be later renamed and/or reclassified. For example, contemplated strains of Anaerostipes hadrus includes those formerly classified as Eubacterium hadrum (Allen-Vercoe et al., Anaerobe. 18(5):523-529 (2011)). In particular embodiments, a contemplated Anaerostipes species strain is an Anaerostipes rhamnosivorans strain.

As used herein, the term “species” refers to a taxonomic entity as conventionally defined by genomic sequence and phenotypic characteristics. A “strain” is a particular instance of a species that has been isolated and purified according to conventional microbiological techniques. Bacterial species and/or strains described herein include those that are live and/or viable, as well as those that are killed, inactivated or attenuated. Additionally, bacterial species and/or strains described herein include vegetative forms and non-spore forming forms of bacteria.

In some embodiments, a bacterial strain of Anaerostipes comprises a 16S rRNA gene sequence having a certain % identity to a reference sequence. rRNA, 16S rDNA, 16S rRNA, 16S, 18S, 18S rRNA, and 18S rDNA refer to nucleic acids that are components of, or encode for, components of the ribosome. There are two subunits in the ribosome termed the small subunit (SSU) and large subunit (LSU). Ribosomal RNA genes (rDNA) and their complementary RNA sequences are widely used for determination of the evolutionary relationships among organisms as they are variable, yet sufficiently conserved to allow cross-organism molecular comparisons. 16S rDNA sequence of the 30S SSU can be used, in embodiments, for molecular-based taxonomic assignments of prokaryotes. For example, 16S sequences may be used for phylogenetic reconstruction as they are general highly conserved but contain specific hypervariable regions that harbor sufficient nucleotide diversity to differentiate genera and species of most bacteria. Although 16S rDNA sequence data has been used to provide taxonomic classification, closely related bacterial strains that are classified within the same genus and species, may exhibit distinct biological phenotypes.

Accordingly, a bacterial strain of the species Anaerostipes rhamnosivorans provided herein includes strains comprising a 16s rRNA gene sequence having a certain % identity to

SEQ ID NO: 1. In some embodiments, the bacterial strain is a strain of the genus Anaerostipes comprising a 16s rRNA gene sequence with at least 93% sequence identity to the polynucleotide sequence of SEQ ID NO: 1. In some embodiments, the bacterial strain comprises a 16s rRNA gene sequence with at least about 93.25%, about 93.5%, about 93.75%, about 94%, about 94.25%, about 94.5%, about 94.75%, about 95%, about 95.25%, about 95.5%, about 95.75%, about 96%, about 96.25%, about 96.5%, about 96.75%, about 97%, about 97.05%, about 97.1%, about 97.15%, about 97.2%, about 97.25%, about 97.3%, about 97.35%, about 97.4%, about 97.45%, about 97.5%, about 97.55%, about 97.6%, about 97.65%, about 97.7%, about 97.75%, about 97.8%, about 97.85%, about 97.9%, about 97.95%, about 98%, about 98.05%, about 98.1%, about 98.15%, about 98.2%, about 98.25%, about 98.3%, about 98.35%, about 98.4%, about 98.45%, about 98.5%, about 98.55%, about 98.6%, about 98.65%, about 98.7%, about 98.75%, about 98.8%, about 98.85%, about 98.9%, about 98.95%, about 99%, about 99.1%, about 99.2%, about 99.3%, about 99.4%, about 99.5%, about 99.6%, about 99.7%, about 99.8%, or about 99.9% identity to the polynucleotide sequence of SEQ ID NO: 1. In a particular embodiment, the bacterial strain comprises a 16s rRNA gene sequence identical to SEQ ID NO: 1. In some embodiments, the sequence identity referred to above is across at least about 70% of SEQ ID NO: 1. In other embodiments, the sequence identity referred to above is across at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of SEQ ID NO: 1.

In some embodiments, a bacterial strain of Anaerostipes rhamnosivorans comprises a genomic sequence (e.g., a whole genome sequence, or fragments or contigs thereof) having a certain % identity to one or more of SEQ ID NOs: 2-52. In some embodiments, an Anaerostipes rhamnosivorans strain comprises a polynucleotide sequence selected from any one of SEQ ID NOs: 2-52, or a nucleotide sequence having at least about 65%, about 70%, about 75%, about 80%, about 85%, about 90%, about 91%, about 92%, about 93%, about 94%, about 95%, about 96%, about 97%, about 98%, or about 99% identity to a polynucleotide sequence selected from any one of SEQ ID NOs: 2-52. In some embodiments, the sequence identity referred to above is across at least about 70% of the bacterial genome. In other embodiments, the sequence identity referred to above is across at least about 75%, 80%, 85%, 90%, 95% or greater than 95% of the bacterial genome. In some embodiments, an Anaerostipes rhamnosivorans strain genome may comprise the polynucleotide sequence of each of SEQ ID NOs: 2-52, or each of a polynucleotide sequence having at least about 65%, about 70%, about 75%, about 80%, about 85%, about 90%, about 91%, about 92%, about 93%, about 94%, about 95%, about 96%, about 97%, about 98%, or about 99% identity to the polynucleotide sequence of each of SEQ ID NOs: 2-52.

In some embodiments, a bacterial strain of Anaerostipes rhamnosivorans comprises a whole genomic sequence having at least about 70% identity across at least 70% of its genome to the sum of all genomic contigs represented by SEQ ID NOs:2-52. In some embodiments, the whole genomic sequence has at least about 75%, 80%, 85%, 90%, 95% or greater than 95% identity to the sum of all genomic contigs represented by SEQ ID NOs:2-52. In some embodiments, the sequence identity referred to above is across at least 75%, 80%, 85%, 90%, 95% or greater than 95% of the whole genomic sequence of the bacterial strain. In some embodiments, a bacterial strain of Anaerostipes rhamnosivorans comprises a whole genomic sequence comprising coding regions having at least about 70% identity across at least 70% of the total coding regions in its genome to the coding regions within the sum of all genomic contigs represented by SEQ ID NOs:2-52. In some embodiments, the coding regions within the whole genomic sequence have at least about 75%, 80%, 85%, 90%, 95% or greater than 95% identity to the coding regions within the sum of all genomic contigs represented by SEQ ID NOs:2-52. In some embodiments, the sequence identity referred to above is across at least 75%, 80%, 85%, 90%, 95% or greater than 95% of the coding regions within the whole genomic sequence of the bacterial strain.

The identity of a bacterial strain of Anaerostipes may be determined by sequence analysis, for example, of the 16s rRNA gene sequence or a genomic sequence (e.g., a whole genome sequence, or fragments or contigs thereof) of the bacterial strain, using any sequencing methods known in the art, including, for example, Sanger sequencing. An example of a sequencing technology useful for identifying strains of Anaerostipes is the Illumina platform. The Illumina platform is based on amplification of DNA on a solid surface (e.g., flow cell) using fold-back PCR and anchored primers (e.g., capture oligonucleotides). For sequencing with the Illumina platform, bacterial DNA is fragmented, and adapters are added to terminal ends of the fragments. DNA fragments are attached to the surface of flow cell channels by capturing oligonucleotides which are capable of hybridizing to the adapter ends of the fragments. The DNA fragments are then extended and bridge amplified. After multiple cycles of solid-phase amplification followed by denaturation, an array of millions of spatially immobilized nucleic acid clusters or colonies of single-stranded nucleic acids are generated. Each cluster may include approximately hundreds to a thousand copies of single-stranded DNA molecules of the same template. The Illumina platform uses a sequencing-by-synthesis method where sequencing nucleotides comprising detectable labels (e.g., fluorophores) are added successively to a free 3′ hydroxyl group. After nucleotide incorporation, a laser light of a wavelength specific for the labeled nucleotides can be used to excite the labels. An image is captured and the identity of the nucleotide base is recorded.

These steps can be repeated to sequence the rest of the bases. Sequencing according to this technology is described in, for example, U.S. Patent Publication Application Nos. 2011/0009278, 2007/0014362, 2006/0024681, 2006/0292611, and U.S. Pat. Nos. 7,960,120, 7,835,871, 7,232,656, and 7,115,200. Another example of a sequencing technology useful for identifying strains of Anaerostipes is SOLiD technology by Applied Biosystems from Life

Technologies Corporation (Carlsbad, Calif). In SOLiD sequencing, bacterial DNA may be sheared into fragments, and adapters may be attached to the terminal ends of the fragments to generate a library. Clonal bead populations may be prepared in microreactors containing template, PCR reaction components, beads, and primers. After PCR, the templates can be denatured, and bead enrichment can be performed to separate beads with extended primers.

Templates on the selected beads undergo a 3′ modification to allow covalent attachment to the slide. The sequence can be determined by sequential hybridization and ligation with several primers. A set of four fluorescently labeled di-base probes compete for ligation to the sequencing primer. Multiple cycles of ligation, detection, and cleavage are performed with the number of cycles determining the eventual read length. Another example of a sequencing technology useful for identifying strains of Anaerostipes is Ion Torrent sequencing. In this technology, bacterial DNA is sheared into fragments, and oligonucleotide adapters are then ligated to the terminal ends of the fragments. The fragments are then attached to a surface, and each base in the fragments is resolvable by measuring the H³⁰ ions released during base incorporation. This technology is described in, for example, U.S. Patent Publication Application Nos. 2009/0026082, 2009/0127589, 2010/0035252, 2010/0137143, and 2010/0188073.

Upon obtaining a polynucleotide sequence of a bacterial strain (e.g., 16s rRNA gene sequence or genomic sequence), sequence identity with a polynucleotide sequence of an Anaerostipes strain may be determined in various ways that are within the skill in the art, e.g., using publicly available computer software such as BLAST, BLAST-2, BLAT (BLAST-like alignment tool), ALIGN or Megalign (DNASTAR) software. BLAST (Basic Local Alignment Search Tool) analysis using the algorithm employed by the programs blastp, blastn, blastx, tblastn and tblastx (Karlin et al., PROC. NATL. ACAD. SCI. USA 87:2264-2268 (1990); Altschul, J. MoL. EvoL. 36, 290-300 (1993); Altschul et al., NUCLEIC ACIDS RES. 25:3389-3402 (1997)) are tailored for sequence similarity searching. For a discussion of basic issues in searching sequence databases see Altschul et al., NATURE GENETICS 6:119-129 (1994) , which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect value (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919), fully incorporated by reference). Four blastn parameters may be adjusted as follows: Q=10 (gap creation penalty); R=10 (gap extension penalty); wink=1 (generates word hits at every wink position along the query); and gapw=16 (sets the window width within which gapped alignments are generated). The equivalent Blastp parameter settings may be Q=9; R=2; wink=1; and gapw=32. Searches may also be conducted using the NCBI

(National Center for Biotechnology Information) BLAST Advanced Option parameter (e.g.: -G, Cost to open gap [Integer]: default=5 for nucleotides/11 for proteins; -E, Cost to extend gap [Integer]: default =2 for nucleotides/1 for proteins; -q, Penalty for nucleotide mismatch [Integer]: default =-3; -r, reward for nucleotide match [Integer]: default =1; -e, expect value [Real]: default =10; -W, wordsize [Integer]: default =11 for nucleotides/28 for megablast/3 for proteins; -y, Dropoff (X) for blast extensions in bits: default =20 for blastn/7 for others; -X, X dropoff value for gapped alignment (in bits): default=15 for all programs, not applicable to blastn; and —Z, final X dropoff value for gapped alignment (in bits): 50 for blastn, 25 for others). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

In a particular embodiment, a bacterial strain of Anaerostipes useful for the compositions and methods provided herein is Anaerostipes rhamnosivorans strain P127-B2a. A deposit of Anaerostipes rhamnosivorans strain P127-B2a was made to DSMZ (Deutsche Sammlung von Mikroorganismen and Zellkulturen GmbH, InhoffenstraBe 7B, 38124 Brunswick, Germany) under the Budapest Treaty on the International Recognition of the Deposit of Microorganisms for the Purposes of Patent Procedure on September 20, 2019.

This deposit was accorded accession number DSM 33275. The 16s rRNA gene sequence of Anaerostipes rhamnosivorans strain P127-B2a is provided herein as SEQ ID NO: 1, and genomic sequences of Anaerostipes rhamnosivorans strain P127-B2a are provided herein as SEQ ID NOs: 2-52.

The terms Anaerostipes rhamnosivorans strain P127-B2a, Anaerostipes sp. P127-B2a,

Anaerostipes P127-B2a, and P127-B2a are used interchangeably herein.

Additional bacterial strains of Anaerostipes provided herein include Anaerostipes strains having a DNA-DNA hybridization (DDH)) value of equal to or greater than about 70% with Anaerostipes rhamnosivorans strain P127-B2a. In particular embodiments, the Anaerostipes rhamnosivorans strain is one having greater than about 70%, about 75%, about 80%, about 85%, about 90%, about 95%, about 96%, about 97%, about 98%, or about 99% DNA-DNA hybridization with Anaerostipes rhamnosivorans strain P127-B2a, or any range between any of the above values. Any method for determining DNA-DNA hybridization values known in the art may be used to assess the degree of DNA-DNA hybridization, including but not limited to the spectrophotometric method for determining renaturation rates described by De Ley et al. Biochem 12 133-142 (1970)), slightly modified in hybridization temperature (Gavini et al., Ecology in Health and Disease 12 40-45 (2001)); and those described by Grimont et al., Curr Microbiol 4, 325-330 (1980) and Rossello-Mora, Molecular Identification, Systematics and Population Structure of Prokaryotes pp. 23-50 (2006). In some embodiments, the degree of DNA-DNA hybridization is determined by digital DNA-DNA hybridization (dDDH) analysis, for example, using the Genome-to-Genome Distance Calculator online tool (see Meier-Kolthoff et al., BMC Bioinformatics 14:60 (2013)). In particular embodiments, the Anaerostipes strain is one having a DDH or dDDH value of equal to or greater than about 70% with Anaerostipes rhamnosivorans strain P127-B2a. In some embodiments, the DDH or dDDH value is greater than about 75%, about 80%, about 85%, about 90%, about 95%, about 96%, about 97%, about 98%, or about 99% with Anaerostipes rhamnosivorans strain P127-B2a, or any range between any of the above values.

Additional bacterial strains of Anaerostipes provided herein include Anaerostipes strains having equal to or greater than 95% average nucleotide identity (ANI) with Anaerostipes rhamnosivorans strain P127-B2a. In some embodiments, the ANI is equal to or greater than about 95%, about 95.5%, about 96%, about 96.5%, about 97%, about 97.5%, about 98%, about 98.5%, about 99%, about 99.5% or 100% with Anaerostipes rhamnosivorans strain P127-B2a, or any range between any of the above values. The average nucleotide identity (ANI) of the shared genes between two strains is known to be a robust means to compare genetic relatedness among strains, and that ANI values of —95% correspond to the 70% DNA—DNA hybridization standard for defining a species. See, e.g., Konstantinidis and Tiedje, Proc Natl Acad Sci USA, 102(7):2567-72 (2005); and Goris et al., Int Syst Evol Microbiol. 57(Pt 1):81-91 (2007); and Jain et al., Nat Commun. 9(1):5114 (2018). In some embodiments, the ANI between two bacterial genomes is calculated from pair-wise comparisons of all sequences shared between any two strains and can be determined, for example, using any of a number of publicly available ANI tools, including but not limited to OrthoANI with usearch (Yoon et al. Antonie van Leeuwenhoek 110:1281-1286 (2017)); ANI Calculator, JSpecies (Richter and Rossello-Mora, Proc Natl Acad Sci USA 106:19126-19131 (2009)); and JSpeciesWS (Richter et al., Bioinformatics 32:929-931 (2016)). Other methods for determining the ANI of two genomes are known in the art. See, e.g., Konstantinidis, K. T. and Tiedje, J. M., Proc. Natl. Acad. Sci. U.S.A., 102: 2567-2572 (2005); and Varghese et al., Nucleic Acids Research, 43(14):6761-6771 (2015); and Jain et al., Nat Commun. 9(1):5114 (2018). In a particular embodiment, the ANI between two bacterial genomes can be determined using an alignment-based method, for example, by averaging the nucleotide identity of orthologous genes identified as bidirectional best hits (BBHs). Protein-coding genes of a first genome (Genome A) and second genome (Genome B) are compared at the nucleotide level using a similarity search tool, for example, NSimScan (Novichkov et al., Bioinformatics 32(15): 2380-23811 (2016). The results are then filtered to retain only the BBHs that display at least 70% sequence identity over at least 70% of the length of the shorter sequence in each BBH pair. The ANI of Genome A to Genome B is defined as the sum of the percent identity times the alignment length for all BBHs, divided by the sum of the lengths of the BBH genes. In another particular embodiment, the ANI between two bacterial genomes can be determined using an alignment-free method, for example, FastANI, which uses alignment-free approximate sequence mapping to assess genomic relatedness. See Jain et al., Nat Commun. 9(1):5114 (2018). FastANI has been demonstrated to reveal clear genetic discontinuity between species, with 99.8% of the total 8 billion genome pairs analyzed conforming to >95% intra-species and <83% inter-species ANI values. Accordingly, in a particular embodiment, a bacterial strain having a genome (Genome A) with equal to or greater than 95% average nucleotide identity (ANI) with the genome of Anaerostipes rhamnosivorans strain P127-B2a (Genome B) is identified as a bacterial strain of the species Anaerostipes rhamnosivorans.

Additional bacterial strains of Anaerostipes provided herein include Anaerostipes strains having equal to or greater than 60% alignment fraction (AF) with Anaerostipes rhamnosivorans strain P127-B2a. In some embodiments, the AF is equal to or greater than about 65%, about 70%, about 75%, about 80%, about 85%, about 90%, about 95%, or 100% with Anaerostipes rhamnosivorans strain P127-B2a, or any range between any of the above values. In some embodiments, the AF is computed by dividing the sum of the lengths of all BBH genes by the sum of the length of all the genes in Genome A. This computation is performed separately in both directions: from Genome A to genome B and from Genome B to Genome A.

In a particular embodiment, an Anaerostipes strain comprises a genome having equal to or greater than about 95% ANI and equal to or greater than 60% AF with the genome of Anaerostipes rhamnosivorans strain P127-B2a. In another particular embodiment, an Anaerostipes strain comprises a genome having equal to or greater than about 96.5% ANI and equal to or greater than 60% AF with the genome of Anaerostipes rhamnosivorans strain

P127-B2a.

Additional bacterial strains of Anaerostipes provided herein include Anaerostipes strains that having the same or approximately the same genome characteristics as Anaerostipes rhamnosivorans strain P127-B2a. Such genome characteristics can include, for example, genome size, G+C content, number of coding sequences, and number of tRNAs. In some embodiments, the Anaerostipes strain comprises a genome of about 3.5 to about 3.7 megabases (Mb) in size. In some embodiments, the Anaerostipes strain comprises a genome of about 3.55 to about 3.65 Mb in size. In some embodiments, the Anaerostipes strain comprises a genome of about 3.55, 3.56, 3.57, 3.58, 3.59, 3.60, 3.61, 3.62, 3.63, 3.64 or about 3.65 Mb in size. In a particular embodiment, the Anaerostipes strain comprises a genome of about 3.59 Mb in size. In some embodiments, the Anaerostipes strain comprises a genome that comprises a G+C content of about 43% to about 46%. In some embodiments, the Anaerostipes strain comprises a genome that comprises a G+C content of about 43.5% to about 45.5%. In some embodiments, the Anaerostipes strain comprises a genome that comprises a G+C content of about 43.6%, 43.7%, 43.8%, 43.9%, 44.0%, 44.1%, 44.2%, 44.3%, 44.4%, 44.5%, 44.6%, 44.7%, 44.8%, 44.9% or about 45.0%. In a particular embodiment, the Anaerostipes strain comprises a genome that comprises a G+C content of about 44.48%. In some embodiments, the Anaerostipes strain comprises a genome that comprises about 3400 to 3600 coding sequences. In some embodiments, the Anaerostipes strain comprises a genome that comprises about 3450 to 3550 coding sequences. In some embodiments, the Anaerostipes strain comprises a genome that comprises about 3465, 3466, 3467, 3468, 3469, 3470, 3471, 3472, 3473, 3474, 3475, 3476, 3477, 3478, 3479, 3480, 3481, 3482, 3483, 3484, or about 3485 coding sequences. In a particular embodiment, the Anaerostipes strain comprises a genome that comprises about 23473 coding sequences. In some embodiments, the Anaerostipes strain comprises a genome that comprises about 40 to 50 tRNA sequences. In some embodiments, the Anaerostipes strain comprises a genome that comprises about 43 to 49 tRNA sequences. In some embodiments, the Anaerostipes strain comprises a genome that comprises about 43, 44, 45, 46, 47, 48, 49 or 50 tRNAs. In a particular embodiment, the Anaerostipes strain comprises a genome that comprises about 46 tRNAs.

Additional bacterial strains of Anaerostipes provided herein include Anaerostipes strains that provide the same or approximately the same pattern as Anaerostipes rhamnosivorans strain P127-B2a when analyzed, for example, by DNA fingerprinting techniques. Any DNA fingerprinting technique known in the art may be used to identify strains of Anaerostipes, including but not limited to, Pulsed Field Gel Electrophoresis (PFGE), ribotyping, Randomly Amplified Polymorphic DNA (RAPD), Amplified Fragment Length Polymorphism (AFLP), Amplified Ribosomal DNA Restriction Analysis (ARDRA), rep-PCR (repetitive element primed PCR, directed to naturally occurring, highly conserved, repetitive DNA sequences, present in multiple copies in the genomes) including Repetitive Extragenic Palindromic PCR (REP-PCR), Enterobacterial Repetitive Intergenic Consensus Sequences-PCR (ERIC-PCR), BOX-PCR (derived from the boxA element), (GTG)₅-PCR, Triplicate Arbitrary Primed PCR (TAP-PCR), Multi-Locus Sequence Analysis (MLSA), Multi-Locus Sequence Typing (MLST), Multiple Locus Variable-number Tandem Repeat Analysis (MLVA) and DNA microarray-based genotyping techniques.

Additional bacterial strains of Anaerostipes provided herein include Anaerostipes strains showing phenotypic similarity to Anaerostipes rhamnosivorans strain P127-B2a.

Phenotypic similarity can be based on, for example, cell shape and size, colony morphology (e.g. size, color and odor of plate colonies), Gram staining, biochemical tests, pH and temperature optima, sugar fermentation, metabolic capabilities (e.g. catalase and/or oxidase negative), chemotaxonomic analysis (e.g. polar lipid and lipoquinone composition; see Tindall et al., Int JSyst Evol Microbiol 58, 1737-1745 (2008)) and/or fatty acid methyl ester (FAME) analysis. In some embodiments, the bacterial strain of Anaerostipes is catalase negative. In some embodiments, the bacterial strain of Anaerostipes is oxidase negative. In some embodiments, the bacterial strain of Anaerostipes is both catalase and oxidase negative.

In some embodiments, a bacterial strain of Anaerostipes is capable of fermenting at least one carbon source selected from dulcitol, fructose (e.g. D-fructose), galactose (e.g. D-galactose), glucosaminitol (e.g. N-acetyl-D-glucosaminitol), glucosamine (e.g. N-acetyl-D-glucosamine, D-glucosamine), glucose (e.g., a-D-glucose), malitol, maltose, mannitol (e.g., D-mannitol), mannose (e.g. D-mannose), palatinose, rhamnose (e.g. D-rhamnose), sorbitol (e.g., D-sorbitol), sorbose (e.g. L-sorbose), sucrose, tagatose (e.g. D-tagatose), turanose, butyric acid (β-hydroxy butyric acid), L-cysteine, L-Aspartic Acid, L-arginine, L-alanine, m-Inositol, X-α-D-glucoside, X-β-D-glucoside, X-β-D-galactoside, X-α-D-mannoside, X-α-D-Glucuronide, and X-α-D-galactoside. In some embodiments, the bacterial strain of Anaerostipes is capable of fermenting each of dulcitol, fructose (e.g. D-fructose), galactose (e.g. D-galactose), glucosaminitol (e.g. N-acetyl-D-glucosaminitol), glucosamine (e.g. N-acetyl-D-glucosamine, D-glucosamine), glucose (e.g., a-D-glucose), malitol, maltose, mannitol (e.g., D-mannitol), mannose (e.g. D-mannose), palatinose, rhamnose (e.g. D-rhamnose), sorbitol (e.g., D-sorbitol), sorbose (e.g. L-sorbose), sucrose, tagatose (e.g. D-tagatose), turanose, butyric acid (β-hydroxy butyric acid), L-cysteine, L-Aspartic Acid, L-arginine, L-alanine, m-Inositol, X-α-D-glucoside, X-β-D-glucoside, X-β-D-galactoside, X-α-D-mannoside, X-α-D-Glucuronide, and X-α-D-galactoside. In certain other embodiments, the bacterial strain of Anaerostipes is not capable of fermenting, or substantially fermenting, mannitol (e.g., D-mannitol) or sorbitol (e.g., D-sorbitol).

In some embodiments, an Anaerostipes bacterial strain useful for the compositions and methods provided herein is capable of producing one or more short-chain fatty acids (SCFAs). In some embodiments, the SCFA is butyric acid. In some embodiments, the Anaerostipes bacterial strain is capable of utilizing one or more short-chain fatty acids (SCFAs) selected from the group consisting of propionic acid and acetic acid.

A contemplated bacterial strain, bacterial strain mixture, or composition may be characterized as having an effect on gene product production, e.g., IL-10, IL-12, or CCL-18 production, in an immune cell, e.g., a macrophage (e.g., a THP-1 macrophage) or PBMC (including lymphocytes (T cells, B cells, NK cells) and monocytes). In vivo, major sources of IL-10 include T helper cells, monocytes, macrophages and dendritic cells, however myriad immune effector cell types are capable of producing IL-10 in certain contexts including B cells, cytotoxic T cells, NK cells, mast cells, and granulocytes like neutrophils and eosinophils. Gene product production, e.g., IL-10, IL-12, or CCL-18, in a macrophage may, for example, be assayed as follows. THP-1 human macrophages are made by culturing the THP-1 human monocyte cell line with phorbol 12-myristate 13-acetate (PMA) for 24 hours, optionally followed by IL-4 and IL-13 as described previously (Genin et al., BMC Cancer 15:577 (2015)). A bacterial strain, bacterial strain mixture, or composition is incubated with THP-1 macrophages in the presence of lipopolysaccharide (LPS) for 24 hours. Gene product production is assessed by measuring the concentration of the gene product, e.g., IL-10, IL-12, or CCL-18, in the cell culture supernatant by ELISA. Gene product production may also be assayed as described in Sudhakaran et al., Genes Nutr., 8(6): 637-48 (2013). Gene product production, e.g., IL-10, IL-12, or CCL-18 production, in a PBMC may, for example, be assayed as follows. Primary PBMCs are isolated from blood samples of donors using a percoll gradient (Sim et al., J. Vis. Exp. (112), e54128 (2016)). A bacterial strain, bacterial strain mixture, or composition is incubated with PBMCs for 24 hours. Gene product production is assessed by measuring the concentration of the gene product, e.g., IL-10, IL-12, or CCL-18, in the cell culture supernatant by ELISA.

For example, in some embodiments, an Anaerostipes (e.g., Anaerostipes rhamnosivorans) bacterial strain, or a bacterial strain mixture or composition comprising an Anaerostipes (e.g., Anaerostipes rhamnosivorans) bacterial strain, increases, or is capable of increasing, production of at least one anti-inflammatory gene product, e.g., an anti-inflammatory cytokine or chemokine, in a cell, tissue, or subject. Exemplary anti-inflammatory gene products include CCL-18, IL-1Ra, IL-2, IL-4, IL-6, IL-10, IL-11, IL-13, IFN-β and TGF-β. For example, in some embodiments, an Anaerostipes bacterial strain (or bacterial strain mixture or composition comprising the Anaerostipes bacterial strain), increases production of CCL-18 and/or IL-10 in a cell, tissue, or subject. In some embodiments, the increased production of an anti-inflammatory gene product, e.g., CCL-18, occurs in a human cell, e.g., a THP-1 macrophage, monocyte, PBMC or moDC. For example, contacting a human cell, e.g., a THP-1 macrophage, PBMC or moDC, with the Anaerostipes bacterial strain (or bacterial strain mixture or composition comprising the Anaerostipes bacterial strain), e.g., by culturing the human cell with the Anaerostipes bacterial strain (or bacterial strain mixture or composition comprising the Anaerostipes bacterial strain) increases production of CCL-18 and/or IL-10 in the cell by at least about 10%, at least about 20%, at least about 30%, at least about 40%, at least about 50%, at least about 75%, at least about 100%, at least about 200%, at least about 300%, at least about 400%, at least about 500%, at least about 750%, at least about 1000%, from about 10% to about 20%, from about 10% to about 50%, from about 10% to about 100%, from about 10% to about 200%, from about 10% to about 500%, from about 10% to about 1000%, from about 20% to about 50%, from about 20% to about 100%, from about 20% to about 200%, from about 20% to about 500%, from about 20% to about 1000%, from about 50% to about 100%, from about 50% to about 200%, from about 50% to about 500%, from about 50% to about 1000%, from about 100% to about 200%, from about 100% to about 500%, from about 100% to about 1000%, from about 200% to about 500%, from about 200% to about 1000%, or from about 500% to about 1000%, relative to a cell (e.g., of the same cell type) that was not contacted, e.g., cultured, with the Anaerostipes bacterial strain (or bacterial strain mixture or composition comprising the Anaerostipes bacterial strain). In some embodiments, the contacting of the human cell with the Anaerostipes bacterial strain (or bacterial strain mixture or composition comprising the Anaerostipes bacterial strain) occurs in vitro. In other embodiments, the contacting of the human cell with the Anaerostipes bacterial strain (or bacterial strain mixture or composition comprising the Anaerostipes bacterial strain) occurs in vivo.

In some embodiments, an Anaerostipes (e.g., Anaerostipes rhamnosivorans) bacterial strain, or a bacterial strain mixture or composition comprising an Anaerostipes (e.g., Anaerostipes rhamnosivorans) bacterial strain, may have anti-and/or pro-inflammatory activity. For example, in some embodiments, an Anaerostipes bacterial strain (or bacterial strain mixture or composition comprising the Anaerostipes bacterial strain), increases, or is capable of increasing, production of at least one pro-inflammatory gene product, e.g., a pro-inflammatory cytokine or chemokine, in a cell, tissue, or subject. Exemplary pro-inflammatory gene products include IL-1-β, IL-4, IL-5, IL-6, IL-8, IL-12 IL-13, IL-17, IL-21, IL-22, IL-23, IL-27, IFN-y, TNF-α, TRAIL, CCL-2, CCL-3, CCL-5, CCL-20, CXCL-5, CXCL-10, CXCL-12, and CXCL-13. For example, in some embodiments, an Anaerostipes bacterial strain (or bacterial strain mixture or composition comprising the Anaerostipes bacterial strain), increases production of IL-1-β, IL-12, and TNF-α in a cell, tissue, or subject. In some embodiments, the increased production of IL-1-β, IL-12, and/or TNF-α occurs in a human cell, e.g., a THP-1 macrophage, monocyte, PBMC or moDC. For example, contacting a human cell, e.g., a THP-1 macrophage, monocyte, PBMC or moDC, with the Anaerostipes bacterial strain (or bacterial strain mixture or composition comprising the Anaerostipes bacterial strain), e.g., by culturing the human cell with the Anaerostipes bacterial strain (or bacterial strain mixture or composition comprising the Anaerostipes bacterial strain) increases production of IL-1-β, IL-12, and/or TNF-α in the cell by at least about 10%, at least about 20%, at least about 30%, at least about 40%, at least about 50%, at least about 75%, at least about 100%, at least about 200%, at least about 300%, at least about 400%, at least about 500%, at least about 750%, at least about 1000%, from about 10% to about 20%, from about 10% to about 50%, from about 10% to about 100%, from about 10% to about 200%, from about 10% to about 500%, from about 10% to about 1000%, from about 20% to about 50%, from about 20% to about 100%, from about 20% to about 200%, from about 20% to about 500%, from about 20% to about 1000%, from about 50% to about 100%, from about 50% to about 200%, from about 50% to about 500%, from about 50% to about 1000%, from about 100% to about 200%, from about 100% to about 500%, from about 100% to about 1000%, from about 200% to about 500%, from about 200% to about 1000%, or from about 500% to about 1000%, relative to a cell (e.g., of the same cell type) that was not contacted, e.g., cultured, with the Anaerostipes bacterial strain (or bacterial strain mixture or composition comprising the Anaerostipes bacterial strain). In some embodiments, the contacting of the human cell with the Anaerostipes bacterial strain (or bacterial strain mixture or composition comprising the Anaerostipes bacterial strain) occurs in vitro. In other embodiments, the contacting of the human cell with the Anaerostipes bacterial strain (or bacterial strain mixture or composition comprising the Anaerostipes bacterial strain) occurs in vivo.

In other embodiments, an Anaerostipes bacterial strain (or bacterial strain mixture or composition comprising the Anaerostipes bacterial strain), reduces or attenuates, or is capable of reducing or attenuating, production of at least one pro-inflammatory gene, e.g., a pro-inflammatory cytokine or chemokine, in a cell, tissue, or subject. In some embodiments, the bacterial strain reduces or attenuates, or is capable of reducing or attenuating, production of at least one pro-inflammatory gene, e.g., a pro-inflammatory cytokine or chemokine, in a cell, tissue, or subject, for example, in the presence of a pro-inflammatory stimulus. Such pro-inflammatory cytokine or chemokine includes IL-113, IL-4, IL-5, IL-6, IL-8, IL-12, IL-13, IL-17, IL-21, IL-22, IL-23, IL-27, IFN, CCL-2, CCL-3, CCL-5, CCL-20, CXCL-5, CXCL-10, CXCL-12, CXCL-13, IFN-y, KC/GRO (keratinocyte chemoattractant (KC) chemokines CXCL1/2, mouse homologues of human growth-regulated oncogenes (GRO)) and TNF-α. For example, in some embodiments, the bacterial strain of Anaerostipes rhamnosivorans reduces or attenuates, or is capable of reducing or attenuating, production of IL-4 and/or KC/GRO in a cell, tissue, or subject. In some embodiments, the reduced or attenuated production of an anti-inflammatory gene product, e.g., IL-4 and/or KC/GRO, occurs in a human cell, e.g., a THP-1 macrophage or monocyte, moDC or a PBMC. For example, contacting a human cell, e.g., a THP-1 macrophage or PBMC, with Anaerostipes rhamnosivorans, e.g., by co-culturing the human cell with Anaerostipes rhamnosivorans, reduces or attenuates production of IL-4 and/or KC/GRO in the cell by at least about 10%, at least about 20%, at least about 30%, at least about 40%, at least about 50%, at least about 75%, from about 10% to about 20%, from about 10% to about 50%, from about 10% to about 100%, from about 20% to about 50%, from about 20% to about 100%, or from about 50% to about 100%, relative to a cell (e.g., of the same cell type) that was not contacted, e.g., cultured with Anaerostipes rhamnosivorans. In some embodiments, the contacting of the human cell with Anaerostipes rhamnosivorans occurs in vitro. In other embodiments, the contacting of the human cell with Anaerostipes rhamnosivorans occurs in vivo.

A contemplated Anaerostipes (e.g., Anaerostipes rhamnosivorans) bacterial strain, or a bacterial strain mixture or composition comprising an Anaerostipes (e.g., Anaerostipes rhamnosivorans)bacterial strain, may (i) reduce or prevent disruption to, or increase, or be capable of reducing or preventing disruption to, or increasing, the integrity of an epithelial barrier (e.g., an epithelial cell monolayer); and/or (ii) increase, or be capable of increasing, production of at least one pro-barrier integrity gene (e.g., ZO-1) in a cell, tissue, or subject. In some embodiments, the epithelial barrier is an intestinal barrier, e.g., an intestinal mucosal barrier. The intestinal epithelium is organized in a single layer of 20 μm, and includes 5 different cell types: enterocytes, endocrine cells, M cells, goblet (mucous) cells and Paneth cells. The enterocytes are the most represented cell type, acting as a physical barrier, inhibiting the translocation of luminal contents in the inner tissues. They are connected by intercellular junctions, characterized by transmembrane proteins that interact with near cells and with intracellular proteins associated with the cytoskeleton. Contemplated bacterial strains, bacterial strain mixtures, or compositions may be characterized by an effect on the integrity of an epithelial barrier, e.g., a HT29MTX-E12 cell monolayer or a Caco-2 monolayer, or any intestinal epithelial cell or cell line monolayer. In certain embodiments, contemplated bacterial strains, bacterial strain mixtures, or compositions may be characterized by an effect on barrier integrity of a human epithelial cell monolayer, e.g., a HT29MTX-E12 cell monolayer or a Caco-2 monolayer, treated with TNF-α. Barrier integrity of a HT29MTX-E12 cell monolayer treated with TNF-α may, for example, be assayed as follows. HT29MTX-E12 cells are seeded into a transwell plate system for 18-21 days to form polarized monolayers as described previously (Hall et al., Journal of Pediatric Surgery 48:353-358 (2013)). A bacterial strain, bacterial strain mixture, or composition is added to the apical layer of the transwell followed by the addition of TNF-α to the basal layer of the transwell, thus modeling an inflamed gut. The monolayer integrity is assessed by measuring the trans-epithelial electrical resistance (TEER) across the cell barrier at 0 and 24 hours after TNF-α addition. Barrier integrity may also be assayed as described in Pontier et al., J. Pharm. Sci., 90(10): 1608-19 (2001).

For example, in certain embodiments, an Anaerostipes (e.g., Anaerostipes rhamnosivorans) bacterial strain provided herein, or a bacterial strain mixture or composition comprising an Anaerostipes (e.g., Anaerostipes rhamnosivorans) bacterial strain provided herein, reduces or prevents disruption to, or increases, or is capable of reducing or preventing disruption to, or increasing, barrier integrity of a mucosal epithelium. For example, an Anaerostipes bacterial strain (or bacterial strain mixture or composition comprising the Anaerostipes bacterial strain) may reduce or prevent disruption of, or increase, barrier integrity of a HT29MTX-E12 cell monolayer, e.g., a HT29MTX-E12 cell monolayer treated with TNF-α. For example, incubation of a contemplated Anaerostipes species strain (or bacterial strain mixture or composition comprising the Anaerostipes bacterial strain) with a HT29MTX-E12 cell monolayer, e.g., a HT29MTX-E12 cell monolayer treated with TNF-α, may reduce or prevent disruption of, or increase, barrier integrity, e.g., as measured by trans-epithelial electrical resistance (TEER), by at least about 10%, at least about 20%, at least about 30%, at least about 40%, at least about 50%, at least about 60%, at least about 70%, at least about 80%, at least about 90%, at least about 100%, from about 20% to about 100%, from about 40% to about 100%, from about 60% to about 100%, from about 80% to about 100%, from about 20% to about 80%, from about 40% to about 80%, from about 60% to about 80%, from about 20% to about 60%, from about 40% to about 60%, or from about 20% to about 40%, relative to a HT29MTX-E12 cell monolayer that was not incubated with the Anaerostipes bacterial strain (or bacterial strain mixture or composition comprising the Anaerostipes bacterial strain).

Also provided herein are strains of the species Faecalibacterium prausnitzii, for example, a strain referred to herein as Faecalibacterium prausnitzii P162-C10a, and compositions, for example, pharmaceutical compositions, comprising such strains. A bacterial strain of the species Faecalibacterium prausnitzii provided herein includes strains comprising a 16s rRNA gene sequence having a certain % identity to SEQ ID NO: 53. In some embodiments, the bacterial strain comprises a 16s rRNA gene sequence with at least about 98.00%, about 98.05%, about 98.1%, about 98.15%, about 98.2%, about 98.25%, about 98.3%, about 98.35%, about 98.4%, about 98.45%, about 98.5%, about 98.55%, about 98.6%, about 98.65%, about 98.7%, about 98.75%, about 98.80%, about 98.85%, about 99%, about 99.1%, about 99.2%, about 99.3%, about 99.4%, about 99.5%, about 99.6%, about 99.7%, about 99.8%, or about 99.9% identity to the polynucleotide sequence of SEQ ID NO:

53. In a particular embodiment, the bacterial strain comprises a 16s rRNA gene sequence identical to SEQ ID NO: 53. In some embodiments, the sequence identity referred to above is across at least about 70% of SEQ ID NO: 53. In other embodiments, the sequence identity referred to above is across at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of SEQ ID NO: 53.

In a particular embodiment, a bacterial strain of Faecalibacterium prausnitzii, provided herein is Faecalibacterium prausnitzii P162-C10a. A deposit of Faecalibacterium prausnitzii P162-Cl0a was made to DSMZ (Deutsche Sammlung von Mikroorganismen and Zellkulturen GmbH, InhoffenstraBe 7B, 38124 Brunswick, Germany) under the Budapest

Treaty on the International Recognition of the Deposit of Microorganisms for the Purposes of Patent Procedure on March 6, 2020. This deposit was accorded accession number DSM 33533. The 16S rDNA sequence of Faecalibacterium prausnitzii P162-C10a is provided as SEQ ID NO: 53.

Additional bacterial strains of the species Faecalibacterium prausnitzii provided herein include Faecalibacterium prausnitzii strains having a DNA-DNA hybridization (DDH) value of equal to or greater than about 70% with Faecalibacterium prausnitzii P162-C10a. In particular embodiments, the Faecalibacterium prausnitzii strain is one having greater than about 70%, about 75%, about 80%, about 85%, about 90%, about 95%, about 98%, or about 99% DNA-DNA hybridization with Faecalibacterium prausnitzii P162-C10a, or any range between any of the above values. In particular embodiments, the Faecalibacterium prausnitzii strain is one having a DDH or dDDH value of equal to or greater than about 70% with Faecalibacterium prausnitzii P162-C10a. In some embodiments, the DDH or dDDH value is greater than about 75%, about 80%, about 85%, about 90%, about 95%, about 98%, or about 99% with Faecalibacterium prausnitzii P162-C10a, or any range between any of the above values.

Additional bacterial strains of the species Faecalibacterium prausnitzii provided herein include Faecalibacterium prausnitzii strains having equal to or greater than 95% average nucleotide identity (ANI) with Faecalibacterium prausnitzii P162-C10a. In some embodiments, the ANI is equal to or greater than about 95%, about 95.5%, about 96%, about 96.5%, about 97%, about 97.5%, about 98%, about 98.5%, about 99%, about 99.5%, or 100% with Faecalibacterium prausnitzii P162-C10a, or any range between any of the above values.

Additional bacterial strains of the species Faecalibacterium prausnitzii provided herein include Faecalibacterium prausnitzii strains having equal to or greater than 60% alignment fraction (AF) with Faecalibacterium prausnitzii P162-C10a. In some embodiments, the AF is equal to or greater than about 65%, about 70%, about 75%, about 80%, about 85%, about 90%, about 95%, or 100% with Faecalibacterium prausnitzii P162-C10a, or any range between any of the above values. In some embodiments, the AF is computed by dividing the sum of the lengths of all BBH genes by the sum of the length of all the genes in Genome A. This computation is performed separately in both directions: from Genome A to genome B and from Genome B to Genome A.

In a particular embodiment, an Faecalibacterium prausnitzii strain comprises a genome having equal to or greater than about 95% ANI and equal to or greater than 60% AF with the genome of Faecalibacterium prausnitzii P162-C10a. In another particular embodiment, an Faecalibacterium prausnitzii strain comprises a genome having equal to or greater than about 96.5% ANI and equal to or greater than 60% AF with the genome of Faecalibacterium prausnitzii P162-C10a.

The present disclosure encompasses derivatives of the disclosed bacterial strains. The term “derivative” includes daughter strains (progeny) or stains cultured (sub-cloned) from the original but modified in some way (including at the genetic level), without negatively altering a biological activity of the strain.

II. Compositions Comprising an Anaerostipes Strain

In another aspect, provided herein are compositions, for example pharmaceutical compositions, comprising a bacterial strain of Anaerostipes (e.g., Anaerostipes rhamnosivorans). In some embodiments, the compositions comprise one or more bacterial strains, including one or more bacterial strains of Anaerostipes. In some embodiments, a composition provided herein comprises a bacterial strain of Anaerostipes rhamnosivorans and does not comprise any other strains or species of bacteria. In other embodiments, the composition comprises a bacterial strain of Anaerostipes rhamnosivorans and at least one or more additional strains or species of bacteria. In some embodiments, the at least one additional strain or species of bacteria in the composition is a bacterial strain of the genus Anaerostipes. For example, the composition may comprise an additional strain of Anaerostipes rhamnosivorans and/or one or more strains of an Anaerostipes species that is not Anaerostipes rhamnosivorans. Exemplary additional Anaerostipes species include

Anaerostipes butyraticus, Anaerostipes caccae, and Anaerostipes hadrus. In other embodiments, the composition may comprise Anaerostipes rhamnosivorans and one or more non Anaerostipes bacterial species. In yet other embodiments, the composition may comprise an Anaerostipes strain selected from Anaerostipes butyraticus, Anaerostipes caccae, and Anaerostipes hadrus, and one or more non Anaerostipes bacterial species.

In some embodiments, the one or more non Anaerostipes bacterial species includes a member of the genus Faecalibacterium, for example, Faecalibacterium prausnitzii. Exemplary strains of Faecalibacterium prausnitzii for use in combination with an Anaerostipes strain described herein includes the strain Faecalibacterium prausnitzii P162-C10a, deposited under accession number DSM 33533. The 16S rDNA sequence of Faecalibacterium prausnitzii P162-Cl0a is provided as SEQ ID NO: 53. Other strains of Faecalibacterium prausnitzii useful for combining with Anaerostipes rhamnosivorans P127-B2a in a composition provided herein include strains comprising a 16S rDNA sequence having at least 98% identity to SEQ ID NO: 53. Additional useful strains include Faecalibacterium prausnitzii strain ATCC 27768.

In some embodiments, the one or more non-Anaerostipes bacterial species includes a member of the genus Christensenella, for example, Christensenella sp. P152-H6d (Christensenella californii). Christensenella sp. P152-H6d is described in U.S. patent application Ser. No. 17/006,430, filed Aug. 28, 2020. A deposit of Christensenella sp. P152-H6d, strain P152-H6d was made to DSMZ (Deutsche Sammlung von Mikroorganismen and Zellkulturen GmbH, InhoffenstraBe 7B, 38124 Brunswick, Germany) under the Budapest Treaty on the International Recognition of the Deposit of Microorganisms for the Purposes of Patent Procedure on Aug. 12, 2019. This deposit was accorded accession number DSM 33237. The 16s rRNA gene sequence of Christensenella sp. P152-H6d, strain P152-H6d is provided herein as SEQ ID NO: 54. In some embodiments, the Christensenella bacterial strain comprises a 16s rRNA gene sequence with at least about 98.00%, about 98.05%, about 98.1%, about 98.15%, about 98.2%, about 98.25%, about 98.3%, about 98.35%, about 98.4%, about 98.45%, about 98.5%, about 98.55%, about 98.6%, about 98.65%, about 98.7%, about 98.75%, about 98.80%, about 98.85%, about 99%, about 99.1%, about 99.2%, about 99.3%, about 99.4%, about 99.5%, about 99.6%, about 99.7%, about 99.8%, or about 99.9% identity to the polynucleotide sequence of SEQ ID NO: 54. In a particular embodiment, the bacterial strain comprises a 16s rRNA gene sequence identical to SEQ ID

NO: 54. In some embodiments, the sequence identity referred to above is across at least about 70% of SEQ ID NO: 54. In other embodiments, the sequence identity referred to above is across at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of SEQ ID NO: 54.

Additional bacterial strains of the species Christensenella californii include Christensenella californii strains having a DNA-DNA hybridization (DDH) value of equal to or greater than about 70% with Christensenella sp. P152-H6d. In particular embodiments, the Christensenella californii strain is one having greater than about 70%, about 75%, about 80%, about 85%, about 90%, about 95%, about 98%, or about 99% DNA-DNA hybridization with Christensenella sp. P152-H6d, or any range between any of the above values. In particular embodiments, the Christensenella californii strain is one having a DDH or dDDH value of equal to or greater than about 70% with Christensenella sp. P152-H6d. In some embodiments, the DDH or dDDH value is greater than about 75%, about 80%, about 85%, about 90%, about 95%, about 98%, or about 99% with Christensenella sp. P152-H6d, or any range between any of the above values.

Additional bacterial strains of the species Christensenella californii include Christensenella californii strains having equal to or greater than 95% average nucleotide identity (ANI) with Christensenella sp. P152-H6d. In some embodiments, the ANI is equal to or greater than about 95%, about 95.5%, about 96%, about 96.5%, about 97%, about 97.5%, about 98%, about 98.5%, about 99%, about 99.5%, or 100% with Christensenella sp. P152-H6d, or any range between any of the above values.

Additional bacterial strains of the species Christensenella californii include Christensenella californii strains having equal to or greater than 60% alignment fraction (AF) with Christensenella sp. P152-H6d. In some embodiments, the AF is equal to or greater than about 65%, about 70%, about 75%, about 80%, about 85%, about 90%, about 95%, or 100% with Christensenella sp. P152-H6d, or any range between any of the above values. In some embodiments, the AF is computed by dividing the sum of the lengths of all BBH genes by the sum of the length of all the genes in Genome A. This computation is performed separately in both directions: from Genome A to genome B and from Genome B to Genome A.

In a particular embodiment, a Christensenella californii strain comprises a genome having equal to or greater than about 95% ANI and equal to or greater than 60% AF with the genome of Christensenella sp. P152-H6d. In another particular embodiment, a Christensenella californii strain comprises a genome having equal to or greater than about 96.5% ANI and equal to or greater than 60% AF with the genome of Christensenella sp. P152-H6d.

In some embodiments, the one or more non-Anaerostipes bacterial species includes (i) a member of the genus Faecalibacterium, for example, Faecalibacterium prausnitzii, and (ii) a member of the genus Christensenella, for example, Christensenella sp. P152-H6d (Christensenella californii).

A contemplated composition or bacterial strain mixture may, e.g., comprise or consist essentially of 2, 3, 4, 5, 6, 7, 8, 9, 10, or more than 10 bacterial strains. In some embodiments, one or more strains of the composition or bacterial strain mixture are vegetative. In some embodiments, all the strains of the composition or bacterial strain mixture are vegetative. For example, in certain embodiments, a disclosed composition or bacterial strain mixture comprises or consists essentially of 2 to 10, 2 to 9, 2 to 8, 2 to 7, 2 to 6, 2 to 5, 2 to 4, or 2 to 3 bacterial strains; or, for example, may comprise or consist essentially of 3 to 10, 3 to 9, 3 to 8, 3 to 7, 3 to 6, 3 to 5 or 3 to 4 bacterial strains; or, for example, may comprise or consist essentially of 4 to 10, 4 to 9, 4 to 8, 4 to 7, 4 to 6 or 4 to 5 bacterial strains; or, for example, may comprise or consist essentially of 5 to 10, 5 to 9, 5 to 8, 5 to 7, 5 to 6, 6 to 10, 6 to 9, 6 to 8, 6 to 7, 7 to 10, 7 to 9, or 7 to 8 bacterial strains; or, for example, may comprise or consist essentially of 8 to 10 or 8 to 9 bacterial strains. In some embodiments, a disclosed composition or bacterial strain mixture comprises or consists essentially of 2 or 3 bacterial strains.

A composition, e.g., a pharmaceutical unit provided herein, may include each bacterial strain at any appropriate ratio, measured either by total mass or by colony forming units of the bacteria. For example, a disclosed pharmaceutical composition or unit may include two strains at a ratio of 0.1:1, 0.2:1, 0.25:1, 0.5:1, 0.75:1, 1:1, 2:1, 3:1, 4:1, 5:1, or 10:1, either by total mass or by colony forming units of the bacteria. For example, a disclosed pharmaceutical composition or unit may include three strains at a ratio of 1:1:1, 1:1:2, 1:1:4, 1:2:1, 1:2:2, 1:2:4, 1:4:1, 1:4:2, 1:4:4, 2:1:1, 2:1:2, 2:1:4, 2:2:1, 2:4:1, 4:1:1, 4:1:2, 4:1:4, 4:2:1, 4:4:1, either by total mass or by colony forming units of the bacteria.

In certain embodiments, the composition comprises a bacterial strain of Anaerostipes rhamnosivorans, and optionally, one or more additional strains or species of bacteria, wherein the composition: (i) increases production of one or more anti-inflammatory gene products, for example CCL-18, IL-1Ra, IL-4, IL-6, IL-10, IL-11, IL-13, and TGF-β, in a human cell, e.g., a THP-1 macrophage or monocyte or a PBMC; and/or (ii) reduces or prevents disruption of, or increases, or is capable of reducing or preventing disruption of, or increasing, barrier integrity of an epithelial cell monolayer, e.g., a HT29MTX-E12 cell monolayer treated with TNF-α.

Excipients

A bacterial strain of Anaerostipes disclosed herein may be combined with pharmaceutically acceptable excipients to form a pharmaceutical composition, which can be administered to a patient by any means known in the art. As used herein, the term “pharmaceutically acceptable excipient” is understood to mean one or more of a buffer, carrier, or excipient suitable for administration to a subject, for example, a human subject, without excessive toxicity, irritation, allergic response, or other problem or complication, commensurate with a reasonable benefit/risk ratio. The excipient (s) should be “acceptable” in the sense of being compatible with the other ingredients of the formulations and not deleterious to the recipient.

Pharmaceutically acceptable excipients include buffers, solvents, dispersion media, coatings, isotonic and absorption delaying agents, and the like, that are compatible with pharmaceutical administration. Pharmaceutically acceptable excipients also include fillers, binders, disintegrants, glidants, lubricants, and any combination(s) thereof. For example, a contemplated composition may comprise a pharmaceutical excipient selected from the group consisting of cellulose, polyvinyl pyrrolidone, silicon dioxide, stearyl fumarate or a pharmaceutically acceptable salt thereof, lactose, starch, glucose, methyl cellulose, ethyl cellulose, hydroxypropyl methyl cellulose, magnesium stearate, mannitol, sorbitol, and any combination(s) thereof. For further examples of excipients, carriers, stabilizers and adjuvants, see, e.g., Handbook of Pharmaceutical Excipients, 8^{th} Ed., Edited by P. J. Sheskey, W. G. Cook, and C.G. Cable, Pharmaceutical Press, London, UK [2017]. The use of such media and agents for pharmaceutically active substances is known in the art.

Stabilized Bacterial Compositions

In certain embodiments, bacterial strains of Anaerostipes described herein may be used in any composition in stabilized form, including, for example, in a lyophilized state (with optionally one or more appropriate cryoprotectants), frozen (e.g., in a standard or super-cooled freezer), spray dried, and/or freeze dried. Stabilized bacteria (e.g. via lyophilization, freezing, spray drying or freeze drying), and in particular, stabilized anaerobic bacteria, may, in certain embodiments, possess advantageous properties over bacteria in culture with respect to administration, e.g., administration of a pharmaceutical composition provided herein. For example, lyophilizing bacterial strains involves a freeze-drying process that removes water from the bacterial cells. The resulting lyophilized bacterial strains may, in certain embodiments, have enhanced stability as compared to bacterial cultures, and thus may be stored for longer periods of time (i.e. extending shelf-life). In addition, in certain embodiments, in stabilized form, dehydrated bacterial cells do not grow or reproduce, but remain viable and may grow and reproduce when rehydrated. In certain embodiments, viability of stabilized anaerobic Anaerostipes bacteria is maintained even when exposed to oxygen, thus facilitating their formulation (for example, into oral dosage forms) and use as a live biotherapeutic product that retains biological activity. Thus, in particular embodiments, the bacterial strains of Anaerostipes described herein are stabilized (e.g. via lyophilization, freezing, freeze-drying or spray-drying), live and viable, and retain some, most, or all of its chemical stability, and/or biological activity upon storage. Stability can be measured at a selected temperature and humidity conditions for a selected time period. Trend analysis can be used to estimate an expected shelf life before a material has actually been in storage for that time period. For live bacteria, for example, stability may be measured as the time it takes to lose 1 log of cfu/g dry formulation under predefined conditions of temperature and/or humidity. Alternatively, stability may be defined in terms of biological function as the time required to measure a decrease in a particular biological function per unit of dry formulation.

In certain embodiments, a pharmaceutical composition or pharmaceutical unit comprising Anaerostipes rhamnosivorans loses at most 0.5 log cfus, 1 log cfus, 1.5 log cfus, 2 log cfus, 2.5 log cfus, 3 log cfus, 3.5 log cfus, 4 log cfus, 4.5 log cfus, 5 log cfus, 5.5 log cfus, 6 log cfus, 6.5 log cfus, 7 log cfus, 7.5 log cfus, 8 log cfus, 8.5 log cfus, 9 log cfus, 9,5 log cfus, or 10 log cfus (total, or per gram of dry formulation) of each bacterial strain present in the pharmaceutical composition or unit upon storage for 1 month, 2 months, 3 months, 4 months, 5 months, 6 months, 7 months, 8 months, 9 months, 10 months 11, months, 12 months, 1 year, 1.5 years, 2 years, 2.5 years, 3 years, 3.5 years, 4 years, 4.5 years, or 5 years at 4° C. or -20° C. For example, the pharmaceutical composition or pharmaceutical unit may lose at most 3 log cfus of each bacterial strain present in the pharmaceutical composition or unit upon storage for 6 months, 1 year, or 2 years at 4° C.

An Anaerostipes bacteria disclosed herein may be combined with one or more cryoprotectants. Exemplary cryoprotectants include fructooligosaccharides (e.g., Raftilose®, fructooligosaccharide derived from inulin), trehalose, maltodextrin, sodium alginate, proline, glutamic acid, glycine (e.g., glycine betaine), mono-, di-, or polysaccharides (such as glucose, sucrose, maltose, lactose), polyols (such as mannitol, sorbitol, or glycerol), dextran, DMSO, methylcellulose, propylene glycol, polyvinylpyrrolidone, non-ionic surfactants such as Tween 80, and/or any combinations thereof.

In certain embodiments, the cryoprotectant comprises Raftilose® (fructooligosaccharide derived from inulin), maltodextrin, alginate, trehalose, and sucrose, or any combinations thereof. In some embodiments, a pharmaceutical composition comprising a bacterial strain of Anaerostipes further comprises sucrose as a cryoprotectant. In some embodiments, a pharmaceutical composition comprising a bacterial strain of Anaerostipes further comprises Raftilose® (fructooligosaccharide derived from inulin), maltodextrin, alginate, trehalose, and sucrose as cryoprotectants. In some embodiments, a pharmaceutical composition comprising a bacterial strain of Anaerostipes further comprises Raftilose® (fructooligosaccharide derived from inulin), maltodextrin, alginate, and trehalose as cryoprotectants.

In some embodiments, a lyophilized powder form of a bacterial strain, as contemplated herein, includes about 10% to about 80% (by weight) of one or more bacterial strains (e.g., one bacterial strain) and about 20% to about 90% (by weight) of a cryoprotectant and/or excipient, such as a cryoprotectant and/or excipient selected from the group consisting of Raftilose° (fructooligosaccharide derived from inulin), maltodextrin, sodium alginate, trehalose, sucrose, water, and/or combinations thereof. For example, 5 mg of contemplated lyophilized powder form of a bacterial strain may include about 0.5 mg to about 1.5 mg of the bacterial strain, about 1.5 mg to about 2.5 mg of the bacterial strain, about 2.5 to about 3.5 mg of the bacterial strain, or about 3.5 mg to about 4.5 mg of the bacterial strain. It can be appreciated that each lyophilized powder form of bacterial strain that may form a component of a disclosed composition may each have different excipients and/or amounts of excipients, as well as a discrete bacterial strain.

A pharmaceutical composition should be formulated to be compatible with its intended route of administration. The bacterial compositions disclosed herein can be prepared by any suitable method and can be formulated into a variety of forms and administered by a number of different means. The compositions can be administered orally, rectally, or enterally, in formulations containing conventionally acceptable carriers, adjuvants, and vehicles as desired. As used herein, “rectal administration” is understood to include administration by enema, suppository, or colonoscopy. A disclosed pharmaceutical composition may, e.g., be suitable for bolus administration or bolus release. In an exemplary embodiment, a disclosed bacterial composition is administered orally.

Solid dosage forms for oral administration include capsules, tablets, caplets, pills, troches, lozenges, powders, and granules. A capsule typically comprises a core material comprising a bacterial composition and a shell wall that encapsulates the core material. In some embodiments the core material comprises at least one of a solid, a liquid, and an emulsion. In some embodiments the shell wall material comprises at least one of a soft gelatin, a hard gelatin, and a polymer. Suitable polymers include, but are not limited to:

cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit®”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In some embodiments at least one polymer functions as a taste-masking agent.

Tablets, pills, and the like can be compressed, multiply compressed, multiply layered, and/or coated. A contemplated coating can be single or multiple. In one embodiment, a contemplated coating material comprises at least one of a saccharide, a polysaccharide, and glycoproteins extracted from at least one of a plant, a fungus, and a microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments a contemplated coating material comprises a protein. In some embodiments a contemplated coating material comprises at least one of a fat and an oil. In some embodiments the at least one of a fat and an oil is high temperature melting. In some embodiments the at least one of a fat and an oil is hydrogenated or partially hydrogenated. In some embodiments the at least one of a fat and an oil is derived from a plant. In some embodiments the at least one of a fat and an oil comprises at least one of glycerides, free fatty acids, and fatty acid esters. In some embodiments a contemplated coating material comprises at least one edible wax. A contemplated edible wax can be derived from animals, insects, or plants. Non-limiting examples include beeswax, lanolin, bayberry wax, carnauba wax, and rice bran wax. Tablets and pills can additionally be prepared with enteric or reverse-enteric coatings.

Alternatively, powders or granules embodying a bacterial composition disclosed herein can be incorporated into a food product. In some embodiments a contemplated food product is a drink for oral administration. Non-limiting examples of a suitable drink include water, fruit juice, a fruit drink, an artificially flavored drink, an artificially sweetened drink, a carbonated beverage, a sports drink, a liquid diary product, a shake, an alcoholic beverage, a caffeinated beverage, infant formula and so forth. Other suitable means for oral administration include aqueous and nonaqueous solutions, emulsions, suspensions and solutions and/or suspensions reconstituted from non-effervescent granules, containing at least one of suitable solvents, preservatives, emulsifying agents, suspending agents, diluents, sweeteners, coloring agents, and flavoring agents.

In certain embodiments, a pharmaceutical composition provided herein includes: (a) an Anaerostipes strain; and (b) a filler (e.g., microcrystalline cellulose, lactose, sucrose, mannitol, or dicalcium phosphate dihydrate), a disintegrant (e.g., polyvinyl pyrrolidone, sodium starch glycolate, starch, or carboxymethyl-cellulose), a flow-aid/glidant (e.g., talc or silica derivatives (e.g., colloidal silica such as Cab-O-Sil or Aerosil)), and a lubricant (e.g., sodium stearyl fumarate, magnesium stearate, calcium stearate, stearic acid, stearic acid salt, talc, liquid paraffin, propylene glycol (PG), PEG 6000, or magnesium/sodium lauryl sulfate).

In certain embodiments, a contemplated pharmaceutical composition includes: (a) an

Anaerostipes strain; and (b) a filler (microcrystalline cellulose), a disintegrant (polyvinyl pyrrolidone), a flow-aid/glidant (silicon dioxide), and a lubricant (sodium stearyl fumarate).

In certain embodiments, a contemplated pharmaceutical composition is formulated as a capsule. In certain embodiments, the capsule is a hydroxypropyl methylcellulose (HPMC) capsule. In certain embodiments, the capsule includes a banding polymer (e.g., hydroxypropyl methylcellulose (HPMC)), and a banding solvent (e.g., water or ethanol). In certain embodiments, the capsule includes two banding solvents, water and ethanol. In certain embodiments the capsule is coated with a reverse enteric coating polymer (e.g., amino methacrylate copolymer), and comprises a surfactant (e.g., sodium lauryl sulfate), a flow-aid/glidant (e.g., silicon dioxide), a lubricant (e.g., stearic acid), an anti-tacking agent (e.g., talc), and a coating solvent (e.g., water). In certain embodiments the capsule is coated with an enteric coating polymer (e.g., poly (methacrylic acid-co-methyl methacrylate)), and further includes a plasticizer (e.g., triethyl citrate), an anti-tacking agent (e.g., talc), a pH adjuster (e.g., ammonia solution), and a coating solvent (e.g., purified water and isopropyl alcohol).

In certain embodiments, a contemplated capsule is a capsule-in-capsule dosage form, which includes an inner capsule and an outer capsule. In certain embodiments, the inner capsule includes one or more lyophilized bacterial strains, a filler (e.g., microcrystalline cellulose, lactose, sucrose, mannitol, dicalcium phosphate dihydrate, or starch), a disintegrant (e.g., polyvinyl pyrrolidone, sodium starch glycolate, or carboxymethyl-cellulose), a flow-aid/glidant (e.g., silicon dioxide, talc, or colloidal silica), and a lubricant (e.g., sodium stearyl fumarate, magnesium stearate, calcium stearate, stearic acid, stearic acid salt, talc, liquid paraffin, propylene glycol (PG), PEG 6000, or magnesium/sodium lauryl sulfate). In certain embodiments, the outer capsule includes one or more lyophilized bacterial strains, a filler (e.g., microcrystalline cellulose, lactose, sucrose, mannitol, dicalcium phosphate dihydrate, or starch). a disintegrant (e.g., polyvinyl pyrrolidone, sodium starch glycolate, or carboxymethyl-cellulose), a flow-aid/glidant (e.g., silicon dioxide, talc, or colloidal silica), and a lubricant (e.g., sodium stearyl fumarate, magnesium stearate, calcium stearate, stearic acid, stearic acid salt, talc liquid paraffin, propylene glycol (PG), PEG 6000, or magnesium/sodium lauryl sulfate).

In certain embodiments, a contemplated capsule is a capsule-in-capsule dosage form, which includes an inner capsule and an outer capsule. In certain embodiments, the inner capsule includes one or more lyophilized bacterial strains, a filler (microcrystalline cellulose), a disintegrant (polyvinyl pyrrolidone), a flow-aid/glidant (silicon dioxide), and a lubricant (sodium stearyl fumarate). In certain embodiments, the outer capsule includes one or more lyophilized bacterial strains, a filler (microcrystalline cellulose), a disintegrant (polyvinyl pyrrolidone), a flow-aid/glidant (silicon dioxide), and a lubricant (sodium stearyl fumarate).

In certain embodiments, a disclosed pharmaceutical unit comprises a dual component capsule. For example, a dual component capsule may comprise an inner capsule, wherein the inner capsule has a reverse enteric polymeric coating, and an outer capsule encapsulating the inner capsule, wherein the outer capsule has an enteric polymeric coating. A contemplated inner and/or outer capsule may comprise a bacterial strain, a bacterial strain mixture. For example, a dual component capsule may comprise an inner capsule having an inner composition comprising a bacterial strain or bacterial strain mixture and one or more pharmaceutical excipients, wherein the inner capsule has a reverse enteric polymeric coating, and an outer capsule encapsulating the inner capsule and an outer composition comprising a bacterial strain or bacterial strain mixture and one or more pharmaceutical excipients, wherein the outer capsule has an enteric polymeric coating. A contemplated inner and/or outer composition may, e.g., comprise an Anaerostipes strain, and optionally one or more additional strains. The inner composition and the outer composition may be the same or different.

A contemplated dual component capsule may include a total of about 5 mg to about 60 mg of the inner and outer composition, e.g., a total of about 5 mg to about 50 mg of the inner and outer composition, a total of about 5 mg to about 15 mg of the inner and outer composition, a total of about 5 mg to about 25 mg of the inner and outer composition, or a total of about 25 mg to about 50 mg of the inner and outer composition. A contemplated dual component capsule may include a total of about 50 mg to about 120 mg of the inner and outer composition, e.g., a total of about 50 mg to about 75 mg of the inner and outer composition, a total of about 60 mg to about 85 mg of the inner and outer composition, a total of about 50 mg to about 95 mg of the inner and outer composition, or a total of about 25 mg to about 110 mg of the inner and outer composition.

In certain embodiments, a disclosed dual component capsule includes an inner capsule with a reverse enteric polymeric coating, and an outer capsule with an enteric polymeric coating. Each respective coating, for example, allows for biphasic release of the capsule's contents (including bacterial strains) at distinct sites along the gastrointestinal tract. For example, it has been determined that the GI tract has several regions sharply demarcated by local pH ranging from 1 to 8.2. The normal pH profile of the GI tract rises and falls between the stomach and the colon with pH ranges of 1-4 in the stomach, 5.5-6.4 in the duodenum, 6.8-8.2 in the ileum, and 5.5-6.5 in the colon. For example, while the distal ileum contains a region where the usual pH is between 6.8 and 8.2, the pH drops sharply from 8.2 to 5.5 after passage through the ileocecal valve into the cecum and ascending colon. The pH gradually rises once again to 8.0 in the progression from proximal to distal colon. Accordingly, in certain embodiments, the enteric polymeric coating of the outer capsule solubilizes in a pH of about 7 to 8, allowing for release in the ileum, and the reverse enteric polymeric coating of the inner capsule solubilizes in a pH of about 6.2 to 6.5, allowing for subsequent release in the colon. In certain embodiments, the outer capsule maintains integrity (e.g., absence of splits, cracks, or rupture of capsule shell) for about 2 hours at pH 1.2 and 37° C. In certain embodiments, the outer capsule maintains integrity (e.g., absence of splits, cracks, or rupture of capsule shell) for about 2 hours at pH 5.5 and 37° C. In certain embodiments, the outer capsule disintegrates within about 1 hour at pH 7.4 and 37° C. In certain embodiments, the inner capsule maintains integrity (e.g., absence of splits, cracks, or rupture of capsule shell) for up to 1 hour at pH 7.4 and 37° C. In certain embodiments, the inner capsule disintegrates within 2 hours at pH 6.5 and 37° C.

In certain embodiments, the inner and/or outer capsule coating is comprised of poly(dl-lactide-co-glycolide, chitosan (Chi) stabilized with PVA (poly-vinylic alcohol), a lipid, an alginate, carboxymethylethylcellulose (CMEC), cellulose acetate trimellitiate (CAT), hydroxypropylmethyl cellulose phthalate (HPMCP), hydroxypropylmethyl cellulose, ethyl cellulose, food glaze, mixtures of hydroxypropylmethyl cellulose and ethyl cellulose, polyvinyl acetate phthalate (PVAP), cellulose acetate phthalate (CAP), shellac, copolymers of methacrylic acid and ethyl acrylate, or copolymers of methacrylic acid and ethyl acrylate to which a monomer of methylacrylate has been added during polymerization. Methylmethacrylates or copolymers of methacrylic acid and methylmethacrylate are available as Eudragit° polymers (Evonik Industries, Darmstadt, Germany). For example, Eudragit® L100 and Eudragit® S100 (anionic copolymers based on methacrylic acid and methyl methacrylate) can be used, either alone or in combination. Eudragit® L100 dissolves at about pH 6 and upwards and comprises between 46.0% and 50.6% methacrylic acid units per g dry substance; Eudragit® S100 dissolves at about pH 7 and upwards and comprises between 27.6% and 30.7% methacrylic acid units per g dry substance. Another exemplary group of encapsulating polymers are the polyacrylic acids Eudragit® L and Eudragit® S which optionally may be combined with Eudragit® RL or RS (copolymers of ethyl acrylate, methyl methacrylate and a low content of methacrylic acid ester with quaternary ammonium groups). These modified acrylic acids are useful since they can be made soluble at a pH of 6 to 7.5, depending on the particular Eudragit chosen, and on the proportion of Eudragit® S to Eudragit® L, RS, and RL used in the formulation. In certain embodiments, a contemplated coating of the inner capsule is comprised of Eudragit EPO® ReadyMix. In certain embodiments, a contemplated coating of the outer capsule is comprised of Eudragit® L100 (methylacrylic acid-methyl methacrylate co-polymer (1:1)) and Eudragit® S100 (methylacrylic acid-methyl methacrylate co-polymer (1:2)). In certain embodiments, a contemplated capsule is suitable for extended or timed release. In certain embodiments, a contemplated inner and/or outer capsule coating further comprises a band/seal, e.g., hypromellose, an opacifier, e.g., titanium dioxide, a plasticizer, e.g. triethyl citrate (TEC) or an anti-tacking agent, e.g. talc.

Further exemplary capsule-in-capsule formulations are described in U.S. Pat. No. 9,907,755.

Unit Dosage Forms

Pharmaceutical compositions comprising an Anaerostipes strain disclosed herein can be presented in a unit dosage form, i.e., a pharmaceutical unit. A composition, e.g., a pharmaceutical unit provided herein, may include any appropriate amount of one or more bacterial strains, measured either by total mass or by colony forming units of the bacteria.

For example, a disclosed pharmaceutical composition or unit may include from about 10³ cfus to about 10¹² cfus, about 10⁶ cfus to about 10¹² cfus, about 10⁷ cfus to about 10¹² cfus, about 10⁸ cfus to about 10¹² cfus, about 10⁹ cfus to about 10¹² cfus, about 10¹⁰ cfus to about 10¹² cfus, about 10¹¹ cfus to about 10¹² cfus, about 10³ cfus to about 10¹¹ cfus, about 10⁶ cfus to about 10¹¹ cfus, about 10⁷ cfus to about 10¹¹ cfus, about 10⁸ cfus to about 10¹¹ cfus, about 10⁹ cfus to about 10¹¹ cfus, about 10¹⁰ cfus to about 10¹¹ cfus, about 10³ cfus to about 10¹⁰ cfus, about 10⁶ cfus to about 10¹⁰ cfus, about 10⁷ cfus to about 10¹⁰ cfus, about 10⁸ cfus to about 10¹⁰ cfus, about 10⁹ cfus to about 10¹⁰ cfus, about 10³ cfus to about 10⁹ cfus, about 10⁶ cfus to about 10⁹ cfus, about 10⁷ cfus to about 10⁹ cfus, about 10⁸ cfus to about 10⁹ cfus, about 10³ cfus to about 10⁸ cfus, about 10⁶ cfus to about 10⁸ cfus, about 10⁷ cfus to about 10⁸ cfus, about 10³ cfus to about 10⁷ cfus, about 10⁶ cfus to about 10⁷ cfus, or about 10³ cfus to about 10⁶ cfus of each bacterial strain, or may include about 10³ cfus, about 10⁶ cfus, about 10⁷ cfus, about 10⁸ cfus, about 10⁹ cfus, about 10¹⁰ cfus, about 10¹¹ cfus, or about 10¹² cfus of a bacterial strain or of each bacterial strain in the composition.

For example, a disclosed pharmaceutical composition or unit may include from about 10³ cfus to about 10¹² cfus, about 10⁶ cfus to about 10¹² cfus, about 10⁷ cfus to about 10¹² cfus, about 10⁸ cfus to about 10¹² cfus, about 10⁹ cfus to about 10¹² cfus, about 10¹⁰ cfus to about 10¹² cfus, about 10¹¹ cfus to about 10¹² cfus, about 10³ cfus to about 10¹¹ cfus, about 10⁶ cfus to about 10¹¹ cfus, about 10⁷ cfus to about 10¹¹ cfus, about 10⁸ cfus to about 10¹¹ cfus, about 10⁹ cfus to about 10¹¹ cfus, about 10¹⁰ cfus to about 10¹¹ cfus, about 10³ cfus to about 10¹⁰ cfus, about 10⁶ cfus to about 10¹⁰ cfus, about 10⁷ cfus to about 10¹⁰ cfus, about 10⁸ cfus to about 10¹⁰ cfus, about 10⁹ cfus to about 10¹⁰ cfus, about 10³ cfus to about 10⁹ cfus, about 10⁶ cfus to about 10⁹ cfus, about 10⁷ cfus to about 10⁹ cfus, about 10⁸ cfus to about 10⁹ cfus, about 10³ cfus to about 10⁸ cfus, about 10⁶ cfus to about 10⁸ cfus, about 10⁷ cfus to about 10⁸ cfus, about 10³ cfus to about 10⁷ cfus, about 10⁶ cfus to about 10⁷ cfus, or about 10³ cfus to about 10⁶ cfus of each bacterial strain, or may include about 10³ cfus, about 10⁶ cfus, about 10⁷ cfus, about 10⁸ cfus, about 10⁹ cfus, about 10¹⁰ cfus, about 10¹¹ cfus, or about 10¹² cfus of a bacterial strain in the composition.

In certain embodiments, a provided pharmaceutical unit comprises at least 1×10³ colony forming units of each bacterial strain (e.g., vegetative bacterial strain), or, at least 1 x 10⁴ colony forming units of bacterial strain (e.g., vegetative bacterial strain), or, at least 1×10⁵ colony forming units of bacterial strain (e.g., vegetative bacterial strain), or, at least 1×10⁶ colony forming units of each bacterial strain (e.g., vegetative bacterial strain), or, at least 1×10⁷ colony forming units of each bacterial strain (e.g., vegetative bacterial strain), or, at least 1×10⁸ colony forming units of each bacterial strain (e.g., vegetative bacterial strain), or, at least 1×10⁹ colony forming units of each bacterial strain (e.g., vegetative bacterial strain).

For example, disclosed compositions (e.g., a pharmaceutical unit such as e.g., a capsule) can include about 1 mg to about 5 mg (e.g., 2 mg to about 4 mg) of a bacterial strain, which can each be present in the unit, e.g., within about 5 mg to about 50 mg of a lyophilized powder form of the bacterial strain. For example, a pharmaceutical unit may comprise a total of about 30 mg to about 70 mg, about 30 mg to about 60 mg, about 30 mg to about 50 mg, about 30 mg to about 40 mg, about 40 mg to about 70 mg, about 40 mg to about 60 mg, about 40 mg to about 50 mg, about 50 mg to about 70 mg, about 50 mg to about 60 mg, about 80 mg to about 100 mg, about 90 mg to about 110 mg, about 100 mg to about 120 mg, or about 110 mg to about 150 mg of lyophilized powder forms of the bacterial strain. In certain embodiments, the pharmaceutical unit comprises a total of about 30 mg, 40 mg, 50 mg, 60 mg, 70 mg, 100 mg, 120 mg, 130 mg, 140 mg, or 150 mg of lyophilized powder form of the bacterial strain.

In certain embodiments, a disclosed composition such as a disclosed pharmaceutical unit may include about 5 to about 50 mg of each lyophilized powder form of a bacterial strain, for example, about 5 to about 45 mg, about 5 to about 40 mg, about 5 to about 35 mg, about 5 to about 30 mg, about 5 to about 25 mg, about 5 to about 15 mg, about 5 to about 10 mg, about 10 to about 50 mg, about 10 to about 35 mg of each lyophilized powder form of a bacterial strain (e.g., a vegetative bacterial strain), about 10 to about 20 mg, about 10 to about 15 mg, or about 15 to about 45 mg of each lyophilized powder form of a bacterial strain (e.g., a vegetative bacterial strain). In certain embodiments, a disclosed pharmaceutical unit comprises about 5, about 10, about 15, about 20, about 25, or about 30 mg of each lyophilized powder form of a bacterial strain (e.g., a vegetative bacterial strain). In certain embodiments, a disclosed pharmaceutical unit includes about 25 to about 50 mg of a lyophilized powder form of one bacterial strain (e.g., vegetative bacterial strain) and about 5 mg to about 10 mg of the remaining lyophilized powder forms of bacterial strains (e.g., vegetative bacterial strains), or about 5 to about 15 mg of one lyophilized powder form of bacterial strain (e.g., vegetative bacterial strain) and about 5 to 10 mg of the remaining lyophilized powder forms of bacterial strains (e.g., vegetative bacterial strains), for example, about 15 mg of one lyophilized powder form of bacterial strain (e.g., vegetative bacterial strain) and about 5 mg of the remaining lyophilized powder forms of bacterial strains (e.g., vegetative bacterial strains), or about 15 mg to about 25 mg of each of two lyophilized powder forms of bacterial strains (e.g., vegetative bacterial strains) and about 5 mg to 10 mg of the remaining lyophilized powder forms of bacterial strains (e.g., vegetative bacterial strains).

In certain embodiments a pharmaceutical composition or unit may include, or may be administered in combination with a prebiotic, i.e., a compound or composition which modifies the growth, maintenance, activity and/or balance of the intestinal micro flora (e.g., can allow for specific changes in the composition and/or activity of the microbiome). Exemplary prebiotics include complex carbohydrates, complex sugars, resistant dextrins, resistant starch, amino acids, peptides, nutritional compounds, biotin, polydextrose, fructooligosaccharide (FOS), galactooligosaccharides (GOS), inulin, lignin, psyllium, chitin, chitosan, chitosanoligosaccharides, lacitol, gums (e.g., guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, isomaltoligosaccharides, and xylooligosaccharides (XOS). Prebiotics can be found in foods (e.g., acacia gum, guar seeds, brown rice, rice bran, barley hulls, chicory root, Jerusalem artichoke, dandelion greens, garlic, leek, onion, asparagus, wheat bran, oat bran, baked beans, whole wheat flour, and banana), and breast milk. Prebiotics can also be administered in other forms (e.g., a capsule or dietary supplement).

### III. Therapeutic Uses

Compositions and methods disclosed herein can be used to treat various forms of inflammatory disorders, gastrointestinal disorders, and/or dysbiosis in a subject. The disclosure provides a method of treating a gastrointestinal disorder, inflammatory disorder, and/or dysbiosis in a subject. A contemplated method comprises administering to the subject an effective amount of a pharmaceutical composition and/or pharmaceutical unit comprising an Anaerostipes bacterial strain disclosed herein (and optionally one or more additional bacterial strains), either alone or in a combination with another therapeutic agent to treat the gastrointestinal disorder, inflammatory disorder, and/or dysbiosis in the subject.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals, e.g., human, a companion animal (e.g., dog, cat, or rabbit), or a livestock animal (for example, cow, sheep, pig, goat, horse, donkey, and mule, buffalo, oxen, or camel)).

It will be appreciated that the exact dosage of a pharmaceutical unit, pharmaceutical composition, or bacterial strain is chosen by an individual physician in view of the patient to be treated, in general, dosage and administration are adjusted to provide an effective amount of the bacterial agent to the patient being treated. As used herein, the “effective amount” refers to the amount necessary to elicit a beneficial or desired biological response. An effective amount can be administered in one or more administrations, applications or dosages and is not intended to be limited to a particular formulation or administration route. As will be appreciated by those of ordinary skill in this art, the effective amount of a pharmaceutical unit, pharmaceutical composition, or bacterial strain may vary depending on such factors as the desired biological endpoint, the drug to be delivered, the target tissue, the route of administration, etc. Additional factors which may be taken into account include the severity of the disease state; age, weight and gender of the patient being treated; diet, time and frequency of administration; drug combinations; reaction sensitivities; and tolerance/response to therapy.

It is understood that a disclosed bacterial strain, bacterial strain mixture, or composition may not require colonization of the gut, e.g., an intestine, of the subject and/or persistence in the subject in order elicit a beneficial or desired biological response. For example, in certain embodiments, a bacterial strain, bacterial strain mixture, or composition colonizes or partially colonizes the gut, e.g. an intestine, of the subject and/or persists in the subject after administration. In certain embodiments, a bacterial strain or bacterial strain mixture does not colonize the gut of the subject and/or persist in the subject after administration.

Inflammatory disorders may be characterized, for example, based on the primary tissue affected, the mechanism of action underlying the disorder, or the portion of the immune system that is misregulated or overactive. Examples of inflammatory disorders include inflammation of the skin, lungs, joints, connective tissue, eyes, nose, bowel, kidney, liver, central nervous system, vascular system, heart, or adipose tissue. In certain embodiments, inflammatory disorders which may be treated include inflammation due to the infiltration of leukocytes or other immune effector cells or mediators thereof into affected tissue. In certain embodiments, inflammatory disorders which may be treated include inflammation mediated by IgA and/or IgE antibodies. Other relevant examples of inflammatory disorders which may be treated by the present disclosure include inflammation caused by infectious agents, including but not limited to viruses, bacteria, fungi, and parasites. In certain embodiments, the inflammatory disorder that is treated is an allergic reaction. In certain embodiments, the inflammatory disorder is an autoimmune disease.

Inflammatory skin disorders include disorders associated with cell proliferation, such as psoriasis, eczema, dermatitis (e.g., eczematous dermatitides, topic and seborrheic dermatitis, allergic or irritant contact dermatitis, eczema craquelee, photoallergic dermatitis, phototoxicdermatitis, phytophotodermatitis, radiation dermatitis, and stasis dermatitis), and acne.

Inflammatory lung disorders include asthma, adult respiratory distress syndrome, bronchitis, pulmonary inflammation, pulmonary fibrosis, and cystic fibrosis (which may additionally or alternatively involve the gastro-intestinal tract or other tissue(s)). Immune mediated inflammatory diseases include systemic lupus erythematosus, systemic vasculitis, Sjogren's syndrome, alopecia areata, and systemic sclerosis. Inflammatory joint disorders include rheumatoid arthritis, seronegative spondyloarthropathies including ankylosing spondylitis, juvenile rheumatoid arthritis, osteoarthritis, gouty arthritis and other arthritic disorders. Inflammatory eye disorders include uveitis (including iritis), conjunctivitis, episcleritis, scleritis, and keratoconjunctivitis sicca. Inflammatory bowel disorders include Crohn's disease, ulcerative colitis, inflammatory bowel disease, and distal proctitis.

Inflammatory disorders of the endocrine system include, but are not limited to, autoimmune endocrinopathies, autoimmune thyroiditis (Hashimoto's disease), Type I diabetes, inflammation in liver and adipose tissue associated with Type II diabetes, and acute and chronic inflammation of the adrenal cortex. Inflammatory disorders of the cardiovascular system include, but are not limited to, coronary infarct damage, peripheral vascular disease, myocarditis, vasculitis, revascularization of stenosis, atherosclerosis, and vascular disease associated with Type II diabetes. Inflammatory disorders of the kidney include, but are not limited to, glomerulonephritis, interstitial nephritis, lupus nephritis, nephritis secondary to

Wegener's disease, acute renal failure secondary to acute nephritis, Goodpasture's syndrome, post-obstructive syndrome and tubular ischemia. Inflammatory disorders of the liver include, but are not limited to, hepatitis (arising from viral infection, autoimmune responses, drug treatments, toxins, environmental agents, or as a secondary consequence of a primary disorder), biliary atresia, primary biliary cirrhosis and primary sclerosing cholangitis. Metabolic disorders with inflammatory etiology include insulin resistance, metabolic syndrome, obesity, Nonalcoholic fatty liver disease (NAFLD), and Nonalcoholic steatohepatitis (NASH). In certain embodiments, the inflammatory disorder is an autoimmune disease, for example, rheumatoid arthritis, lupus, alopecia, autoimmune pancreatitis, Celiac disease, Behcet's disease, Cushing syndrome, and Grave's disease. In certain embodiments, the inflammatory disorder is a rheumatoid disorder, for example, rheumatoid arthritis, juvenile arthritis, bursitis, spondylitis, gout, scleroderma, Still's disease, and vasculitis. Additional exemplary inflammatory disorders include eosinophilic esophagitis and eosinophilic gastroenteritis.

Gastrointestinal disorders include for example, inflammatory bowel disease (IBD), Crohn's disease (CD), ulcerative colitis (UC), ulcerative proctitis, microscopic colitis, irritable bowel syndrome (IBS; e.g., IBS-c, IBS-m, or IBS-d), functional diarrhea, functional constipation, coeliac disease, radiation enteritis, Clostridium difficile (C. difficile) infection (CDI), recurrent C. difficile infection (rCDI), C. difficile associated diarrheal disease (CDAD), colitis (e.g., infectious, ischemic, indeterminate, or radiation colitis), ulcers (including gastric, peptic, and duodenal ulcers), gastroesophageal reflux disease (GERD), pouchitis, gastroenteritis, pancreatitis, mucositis (e.g., oral mucositis, gastrointestinal mucositis, nasal mucositis and proctitis), necrotizing enterocolitis, esophagitis, non-ulcer dyspepsia, chronic intestinal pseudo-obstruction, functional dyspepsia, colonic pseudo-obstruction, duodenogastric reflux, ileus inflammation, post-operative ileus, heartburn (high acidity in the GI tract), constipation (e.g., constipation associated with use of medications such as opioids, osteoarthritis drugs, osteoporosis drugs, post-surgical constipation, or constipation associated with neuropathic disorders), hemorrhoids, diverticular disease, chronic pancreatitis, blind loop syndrome, gastroparesis (including diabetic and/or idiopathic), diarrhea, dysphagia, fecal incontinence, short bowel syndrome (SBS), intestinal ischemia, infant regurgitation, infant rumination syndrome, cyclic vomiting syndrome, globus, volvulus, cancers of the gastrointestinal tract, and gastrointestinal allergies. It is contemplated that compositions and methods disclosed herein can be used to treat any functional gastrointestinal disorder, including, for example, a disorder mediated by or otherwise associated with a brain-gut interaction.

Inflammatory Bowel Disease or IBD is used interchangeably herein to refer to diseases of the bowel that cause inflammation and/or ulceration and includes without limitation Crohn's disease and ulcerative colitis. Crohn's disease (CD) and ulcerative colitis (UC) are chronic inflammatory bowel diseases of unknown etiology.

Ulcerative colitis (UC) afflicts the large intestine. The course of the disease may be continuous or relapsing, mild or severe. The earliest lesion is an inflammatory infiltration with abscess formation at the base of the crypts of Lieberkuhn. Coalescence of these distended and ruptured crypts tends to separate the overlying mucosa from its blood supply, leading to ulceration. Symptoms of the disease include cramping, lower abdominal pain, rectal bleeding, and frequent, loose discharges consisting mainly of blood, pus and mucus with scanty fecal particles. A total colectomy may be required for acute, severe or chronic, unremitting ulcerative colitis.

Crohn's disease, unlike ulcerative colitis, can affect any part of the bowel. The most prominent feature Crohn's disease is the granular, reddish-purple edematous thickening of the bowel wall. With the development of inflammation, these granulomas often lose their circumscribed borders and integrate with the surrounding tissue. Diarrhea and obstruction of the bowel are the predominant clinical features. As with ulcerative colitis, the course of Crohn's disease may be continuous or relapsing, mild or severe, but unlike ulcerative colitis, Crohn's disease is not curable by resection of the involved segment of bowel. Most patients with Crohn's disease require surgery at some point, but subsequent relapse is common and continuous medical treatment is usual.

Generally, dysbiosis refers to a state of the microbiota or microbiome of the gut or other body area, including, e.g., mucosal or skin surfaces (or any other microbiota niche) in which the normal diversity and/or function of the ecological network is disrupted. Any disruption from a typical (e.g., ideal) state of the microbiota can be considered a dysbiosis, even if such dysbiosis does not result in a detectable decrease in health. This state of dysbiosis may be unhealthy (e.g., result in a diseased state), or it may be unhealthy under only certain conditions, or it may prevent a subject from becoming healthier. Dysbiosis may be due to a decrease in diversity of the microbiota population composition, the overgrowth of one or more population of pathogens (e.g., a population of pathogenic bacteria) or pathobionts, the presence of and/or overgrowth of symbiotic organisms able to cause disease only when certain genetic and/or environmental conditions are present in a patient, or the shift to an ecological network that no longer provides a beneficial function to the host and therefore no longer promotes health. A distal dysbiosis includes, but is not limited to, a dysbiosis outside of the lumen of the gastrointestinal tract.

It is contemplated that dysbiosis may include infection with a pathogenic bacterium of a genus selected from the group consisting of Yersinia, Vibrio, Treponema, Streptococcus, Staphylococcus, Shigella, Salmonella, Rickettsia, Orientia, Pseudomonas, Neisseria, Mycoplasma, Mycobacterium, Listeria, Leptospira, Legionella, Klebsiella, Helicobacter, Haemophilus, Francisella, Escherichia, Ehrlichia, Enterococcus, Coxiella, Corynebacterium, Clostridium, Chlamydia, Chlamydophila, Campylobacter, Burkholderia, Brucella, Borrelia, Bordetella, Bifidobacterium, and Bacillus. Further examples of pathogenic bacteria include Aeromonas hydrophila, Campylobacter fetus, Plesiomonas shigelloides, Bacillus cereus, Campylobacter jejuni, Clostridium botulinum, Clostridium difficile, Clostridium perfringens, enteroaggregative Escherichia coli, enterohemorrhagic Escherichia coli, enteroinvasive Escherichia coli, enterotoxigenic Escherichia coli (LT or ST), Escherichia coli 0157:H7, Helicobacter pylori, Lysteria monocytogenes, Plesiomonas shigelloides, Salmonella typhi, Staphylococcus aureus, Vibrio cholerae, Vibrio parahaemolyticus, Vibrio vulnificus, Yersinia enterocolitica, carbapenem-resistant Enterobacteriaceae (CRE), extended spectrum beta-lactam resistant Enterococci (ESBL), vancomycin-resistant Enterococci (VRE), and multi-drug resistant bacteria.

Compositions and methods disclosed herein can also be used to treat cancer in a subject. A contemplated method comprises administering to the subject an effective amount of a pharmaceutical composition and/or pharmaceutical unit comprising an Anaerostipes bacterial strain disclosed herein (and optionally one or more additional bacterial strains), either alone or in a combination with another therapeutic agent to treat the cancer in the subject. Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma). In certain embodiments, the cancer is colorectal cancer (CRC).

In other embodiments, the compositions and methods disclosed herein may also be useful for preventing one or more of the above diseases or conditions, when administered as vaccine compositions. In certain such embodiments, the bacterial strains provided herein are viable. In certain such embodiments, the bacterial strains are capable of at least partially or totally colonizing the gastrointestinal tract, e.g., the intestine. In certain such embodiments, the bacterial strains of the invention are viable and capable of at least partially or totally colonizing the gastrointestinal tract, e.g., the intestine. In other certain such embodiments, the bacterial strains of the invention may be killed, inactivated or attenuated. In certain such embodiments, the compositions may comprise a vaccine adjuvant. In certain embodiments, the compositions are for administration via injection, such as via subcutaneous injection.

### IV. Combination Therapy

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. In certain embodiments, a side effect of a first and/or second treatment is reduced because of combined administration.

In certain embodiments, a method or composition described herein is administered in combination with one or more additional therapies. In certain embodiments, a contemplated additional therapy may include an aminosalicylate, a corticosteroid, a Tumor Necrosis Factor (TNF) antagonist, linaclotide, an antibiotic, or an immunosuppressive agent (e.g., azathioprine, 6-mercaptopurine, cyclosporine, methotrexate, or tacrolimus (Prograf)). In certain embodiments, a contemplated additional therapy may include a biologic agent (e.g., infliximab (Remicade), adalimumab (Humira), certolizumab pegol (Cimzia), golimumab

(Simponi), or etanercept (Enbrel)). It is contemplated that a subject treated with a disclosed method or composition may have had an inadequate response to a previous administration of a therapy, e.g., a previous administration of an aminosalicylate, a corticosteroid, or a biologic agent.

Further therapeutic agents suitable for use in combination therapy with a pharmaceutical composition or unit described herein include proton pump inhibitors (such as pantoprazole (Protonix), lansoprazole (Prevacid), esomeprazole (Nexium), omeprazole (Prilosec), and rabeprazole), H2 blockers (such as cimetidine (Tagamet), ranitidine (Zantac), famotidine (Pepcid), and nizatidine (Axid)), prostaglandins (such as misoprostoL (Cytotec)), sucralfate, and antacids.

In certain embodiments, a pharmaceutical composition or unit may include, or be administered in combination with, a corticosteroid. Corticosteroids are a class of chemicals that includes steroid hormones naturally produced in the adrenal cortex of vertebrates and analogues of these hormones that are synthesized in laboratories. Corticosteroids are involved in a wide range of physiological processes, including stress response, immune response, and regulation of inflammation, carbohydrate metabolism, protein catabolism, blood electrolyte levels, and behavior. Exemplary corticosteroids include betamethasone, budesonide, cortisone, dexamethasone, hydrocortisone, methylprednisolone, prednisolone, prednisone, or deflazacort. It is contemplated that a subject treated with a disclosed method or composition may have had an inadequate response to a previous administration of a corticosteroid.

In certain embodiments, a pharmaceutical composition or unit may include, or be administered in combination with, an aminosalicylate. Exemplary aminosalicylate include 4-Aminosalicylic acid, Balsalazide, Olsalazine, Sulfasalazine, and Mesalazine (5-Aminosalicylic acid). It is contemplated that a subject treated with a disclosed method or composition may have had an inadequate response to a previous administration of mesalamine, for example, a previous administration of ≥2.4g/day mesalamine orally for at least 8 weeks.

In certain embodiments, a pharmaceutical composition or unit may include, or be administered in combination with, a Tumor Necrosis Factor (TNF) antagonist. Exemplary TNF antagonists include infliximab (Remicade), adalimumab (Humira), certolizumab pegol (Cimzia), golimumab (Simponi), etanercept (Enbrel), thalidomide (Immunoprin), lenalidomide (Revlimid), pomalidomide (Pomalyst, Imnovid), xanthine derivatives (e.g., pentoxifylline), and bupropion. It is contemplated that a subject treated with a disclosed method or composition may have had an inadequate response to a previous administration of a TNF antagonist.

In certain embodiments, a pharmaceutical composition or unit may include, or be administered in combination with, an integrin α₄β₇ antagonist, e.g., vedolizumab. It is contemplated that a subject treated with a disclosed method or composition may have had an inadequate response to a previous administration of an integrin a₄β₇ antagonist.

In certain embodiments, a pharmaceutical composition or unit may include, or be administered in combination with, an anti-bacterial agent, e.g., an antibiotic. A disclosed method may comprise pretreatment with an antibiotic, e.g., administration of an antibiotic to a subject prior to administration of a disclosed pharmaceutical composition or unit. Exemplary antibiotics for use in combination therapy include vancomycin, metronidazole, gentamicin, colistin, fidaxomicin, telavancin, oritavancin, dalbavancin, daptomycin, cephalexin, cefuroxime, cefadroxil, cefazolin, cephalothin, cefaclor, cefamandole, cefoxitin, cefprozil, ceftobiprole, cipro, Levaquin, floxin, tequin, avelox, norflox, tetracycline, minocycline, oxytetracycline, doxycycline, amoxicillin, ampicillin, penicillin V, dicloxacillin, carbenicillin, methicillin, ertapenem, doripenem, imipenem/cilastatin, meropenem, amikacin, kanamycin, neomycin, netilmicin, tobramycin, paromomycin, cefixime, cefdinir, cefditoren, cefoperazone, cefotaxime, ceftazidime, ceftibuten, ceftizoxime, ceftriaxone, cefoxotin, and/or streptomycin.

In certain embodiments, a pharmaceutical composition or unit may include, or be administered in combination with, an anti-fungal or anti-viral agents. Exemplary anti-viral agents include abacavir, acyclovir, adefovir, amprenavir, atazanavir, cidofovir, darunavir, delavirdine, didanosine, docosanol, efavirenz, elvitegravir, emtricitabine, enfuvirtide, etravirine, famciclovir, foscarnet, fomivirsen, ganciclovir, indinavir, idoxuridine, lamivudine, lopinavir, maraviroc, MK-2048, nelfinavir, nevirapine, penciclovir, raltegravir, rilpivirine, ritonavir, saquinavir, stavudine, tenofovir trifluridine, valaciclovir, valganciclovir, vidarabine, ibacitabine, amantadine, oseltamivir, rimantidine, tipranavir, zalcitabine, zanamivir and zidovudine. Exemplary anti-fungal agents include natamycin, rimocidin, filipin, nystatin, amphotericin B, candicin, and hamycin, miconazole, ketoconazole, clotrimazole, econazole, omoconazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, terconazole, and albaconazole, abafungin, terbinafine, naftifine, butenafine, anidulafungin, caspofungin, micafungin, polygodial, benzoic acid, ciclopirox, tolnaftate, undecylenic acid, flucytosine or 5-fluorocytosine, griseofulvin, and haloprogin.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure. For example, it will be appreciated that all features described and depicted herein can be applicable to all aspects of the disclosure described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present disclosure remains operable. Moreover, two or more steps or actions may be conducted simultaneously.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

### EXAMPLES

The following Examples are merely illustrative and are not intended to limit the scope or content of the disclosure in any way.

### Example 1

### Isolation and Purification of Anaerostipes rhamnosivorans

1.1 Source. Isolate P127-B2a was isolated from the stool sample of a healthy human donor. The donor underwent comprehensive clinical and laboratory testing to confirm healthy status including screening for infectious agents to minimize risk of transmissible infection. Serology screening included HIV-1/HIV-2 (IgG and EIA), HTLV-I and HTLV-II (Ab), Hepatitis A virus (IgM), Hepatitis B virus (HBSAg, anti-HBc IgG and IgM), Hepatitis C virus (anti-HCV IgG), Treponema pallidum (EIA, or RPR if EIA is positive), Strongyloides stercoralis Ab, CMV Viral Load, and EBV Viral Load. Stool screening included Clostridium difficile toxin AB (PCR), routine bacterial culture for enteric pathogens (with enrichment) including H. pylori EIA, Salmonella, Shigella, Yersinia, Campylobacter, and Vibrio, E. coli 0157 (perform E. coli 0157 culture, if stxl/2 EIA +ve), Shiga-like toxins stx1/2 (Shigella) EIA, Culture-based assays for vancomycin-resistant Enterococcus (VRE), extended spectrum beta-lactamase (ESBL) producers, carbapenem-resistant Enterobacteriaceae (CRE), and methicillin-resistant Staphylococcus aureus (MRSA), Giardia antigen (EIA), Cryptosporidium antigen (EIA), Cyclospora, Isospora, and Microsporidia (Microscopic observation with acid fast stain), Ova and Parasites (Microscopic observation), Rotavirus (EIA), Norovirus GI/GII (RT-PCR), and Adenovirus 40,41 EIA.

1.2 Isolation and Purification. Dilutions of donor samples were plated on isolation media. Colonies were picked from isolation media agar plates (YCFAC, BHI supplemented with Vitamin K and Hemin, TSA supplemented with 5% sheep blood, BUA OxyPras) into a 96-well microtiter plate containing 200 μl of BHI+Hemin+Vitamin K. Once growth was observed visually in the 96-well microtiter plate, 20 μl of culture from each well of the 96-well microtiter plate was transferred into 96-well Deep-Well plate containing 1 ml BHI+Hemin+Vitamin K, followed by incubation at 37° C. After visually detecting growth, 1 ml of 50% glycerol was added to each well, and 600 μl of the mix was transferred into a Thermo Fisher Matrix tube plate. Individual cultures were subsequently plated on isolation media for conformation of colony morphology uniformity. Colonies were observed after 24 hour incubation at 37° C., appearing white and round. Individual colonies were picked for identification by 16S sequencing and replated on YCFAC. After colonies were visible and monomorphology was observed, a single colony was inoculated into 6 ml of YCFAC media. Once the liquid culture became turbid, a matrix plate was prepared by adding 6 ml 50% glycerol to the liquid culture and aliquoting 120 μl per matrix tube. Purity was confirmed by plating from one of the prepared matrix vials onto a BUA OxyPras plate and testing of single colonies by 16S sequencing.

### Example 2

### Taxonomic Characterization of Isolate P127-B2a

2.1 16S Sequencing and Phylogenetic Analysis.

A taxonomic characterization of purified isolate P127-B2a was performed using full length 16S rRNA gene sequencing data. Homology searches were performed against existing publicly available strains present in the National Center for Biotechnology Information (NCBI) taxonomy database and the SILVA ribosomal RNA database (Max Planck Institute for Marine Microbiology and Jacobs University, Bremen, Germany).

2.1.1 16S rRNA gene sequencing. 50 μ1 of a liquid culture of isolate P127-B2a was denatured at 95° C. for 10 minutes. The denatured sample was utilized as a template to PCR amplify the 16S gene by using 16S rRNA primers 27F and 1492R. Sanger sequencing was performed (Elim Biopharm, Hayward, CA) using a set of 4 primers (27F, 1492R, 515F, and 907R) to recover a near full length 16S rRNA gene fragment (SEQ ID NO: 1). The four amplicons were assembled into a single contiguous sequence using DNAbaser (Heracle BioSoft S. R. L., Arges, Romania) which was then searched against the NCBI database using BLASTn.

2.1.2 Phylogenetic analysis. The P127-B2a contig and its close relatives from the NCBI 16S database including the outgroup were next searched against the ARB SILVA database using Alignment (SINA v1.2.11), Classification and Tree service. For search and classification, sequences bearing a minimum of 92 percent identity (15 total sequences) were utilized to classify P127-B2a. RaxML (Randomized Axelerated Maximum Likelihood) was used for performing the maximum likelihood search for building the phylogenetic tree (General Time Reversible (GTR+gamma) model with gamma as rate model for likelihoods).

Preliminary search against the 16S rRNA gene database on NCBI yielded a closest match of 96.94% over 98% of the sequence length of 1429 bps (SEQ ID NO:1). This match was from the species Anaerostipes caccae. The next closest match over the entire length was of 94.6% identity from the species Anaerostipes hadrus.

The selected sequences from NCBI database along with the P127-B2a 16S contig were searched against the ARB SILVA database. The closest match was found to be of 97.5% identity from the an unidentified Anaerostipes species named Clostridiales bacterium VE202-09. The reference 16S rRNA gene of length was 1515 bp (ARB ID: BAHWO₂₀₀₀₀₆₄). The identity dropped to 97.41% over the SEED alignment.

The P127-B2a contig was then searched against the Nucleotide collection (nt) database on NCBI using BLASTn, yielding a match of 100% over 100% of the sequence length of SEQ ID NO:1, corresponding to Anaerostipes rhamnosivorans strain ly2 (Sequence

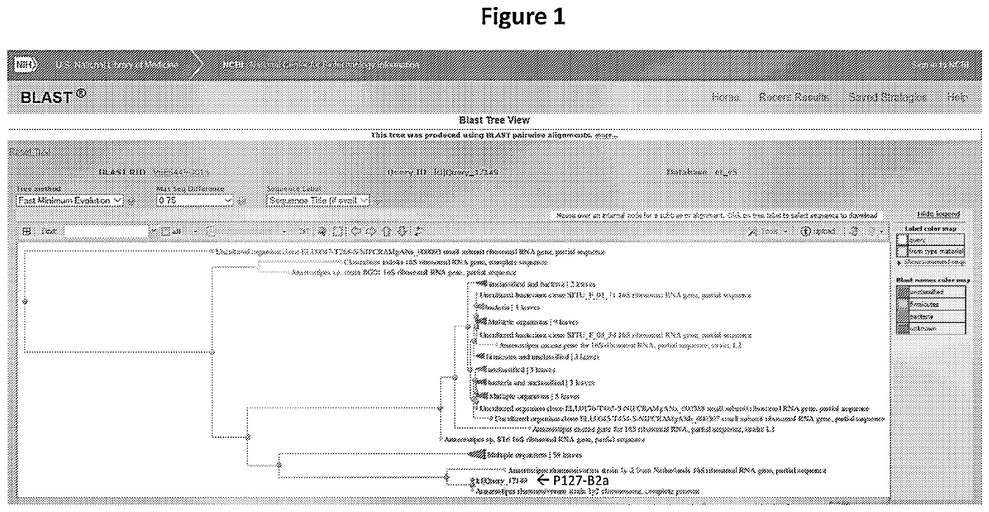

ID: CP040058.1:14645 to 16073). A distance tree based on pairwise comparisons of the search results is provided in FIG. 1.

A summary of the taxonomic identification results is provided in Table 1.

TABLE 1
Taxonomic Identification Result Summary

| | Isolate 16S | |
|---|---|---|
| | sequence | Percent |
| Database match | coverage | Identity |
| Anaerostipes rhamnosivorans strain 1y2 | 100% | 100% |
| Anaerostipes caccae strain L1-92 | 98% | 96.94% |
| Anaerostipes hadrus strain DSM 3319 | 100% | 94.06% |
| Anaerostipes butyraticus strain JCM 17466 | 100% | 93.38% |

2.2 Whole Genome Sequencing and Analysis.

2.2.1 Sequencing. DNA extraction, sequencing, quality filtering, assembly and annotation was performed by Corebiome, Inc. (Minneapolis, MN). DNA was extracted from isolate P127-B2a with MO Bio PowerFecal (Qiagen) automated for high throughput on QiaCube (Qiagen), with bead beating in 0.1 mm glass bead plates. Samples were quantified with Qiant-iT Picogreen dsDNA Assay (Invitrogen). Libraries were prepared with a proprietary procedure adapted for the Nextera Library Prep kit (Illumina) and sequenced on an Illumina NextSeq using single-end 1×150 reads with a NextSeq 500/550 High Output v2 kit (Illumina). DNA sequences were filtered for low quality (Q-Score<20) and length (<50), and adapter sequences were trimmed using cutadapt v.1.15 (Martin, EMBnet Journal, [S.1.], v. 17, n. 1, p. pp. 10-12, (2011)).

2.2.2 Assembly and Annotation. Sequences were assembled using SPAdes v3.11.0 (Bankevich et al., J Comput Biol. 19(5):455-477 (2012)). Protein annotation was performed with Prokka v 1.12 (Seemann, Bioinformatics 30(14):2068-2069 (2014)) on contigs over 1,000 bases in length.

2.2.3 Quality Assessment. Sequencing quality was determined by inspecting quality scores generated by FASTQC, with bases of low quality indicated by scores less than 20. Assembly quality metrics were generated by QUAST v.4.5 (Gurevich et al., Bioinformatics 29(8):1072-1075 (2013)).

2.2.4 Taxonomy. Taxonomic identities were made using appropriate score cut-offs on average nucleotide identity and alignment fraction scores.

2.2.5 Genome Characteristics. Intrinsic properties of the isolate P127-B2a genome assembly were compared with that of the closest Anaerostipes reference, Anaerostipes caccae (accession no. NZ_ABAX00000000) and summarized in Table 2 below.

TABLE 2
Characteristics of P127-B2a PI assemblies.

| | |
|---|---|
| Genome Size | 3.59 |
| (Mb) | |
| Contigs | 51 |
| G + C Content | 44.48 |
| # CDS | 3473 |
| # tRNA | 46 |

PI = primary isolate;
Mb = megabase pairs;
CDS = coding sequence,
tRNA = transfer ribonucleic acid

2.2.6 Genome wide similarity across P127-B2a and other members of Anaerostipes.

The PI genome was compared against other members of Anaerostipes to measure the extent of genomic similarity, in particular, average nucleotide identity (ANI). The results are summarized in Table 3 below.

TABLE 3
Average Nucleotide Identity (ANI) and Alignment Fraction
(AF) of P127-B2a compared to other Anaerostipes spp.

| Isolate ID (S) | Reference Species (R) | ANI S→R |
|---|---|---|
| P127-B2a | Anaerostipes rhamnosivorans | 99.91 |
| P127-B2a | Anaerostipes caccae | 81.09 |
| P127-B2a | Anaerostipes hadrus | 72.70 |

### Example 3

### Phenotypic Characterization of Isolate P127-B2a

P127-B2a cells are non-motile and obligate anaerobes, and are catalase and oxidase negative. P127-B2a was evaluated for its ability to utilize different carbon sources and nitrogen sources, as well as its ability to grow in a wide range of pH using Phenotypic Microarrays (Biolog, Hayward, CA). P127-B2a was able to utilize dulcitol, D-fructose, D-galactose, N-acetyl-D-glucosaminitol, N-acetyl-D-glucosamine, D-glucosamine, a-D-glucose, malitol, maltose, D-mannose, palatinose, D-rhamnose, L-sorbose, sucrose, D-tagatose, turanose, β-hydroxy butyric acid, L-cysteine, L-Aspartic Acid, L-arginine, L-alanine, m-Inositol, X-α-D-glucoside, X-β-D-glucoside, X-βD-galactoside, X-α-D-mannoside, X-α-D-Glucuronide, and X-α-D-galactoside. The P127-B2a strain was able to grow in pH ranging from 5 to 8.5, though optimal growth was observed at pH 7.

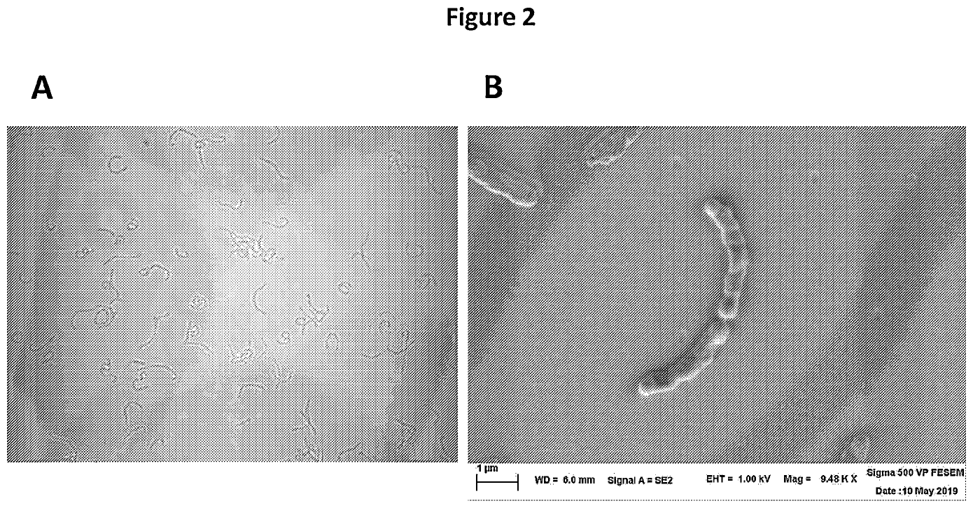

P127-B2a cells were prepared for imaging by electron microscopy. Cells were washed two times in PBS and fixed in 4% paraformaldehyde at room temperature for 30 minutes. Fixed cells were washed two times in PBS, then resuspended in sterile water. Twenty-five microliters of sample were applied to an ITO Coated Cover Slip, 22×22 mm Thickness #1, 30-60 Ohms Resistivity (SPI Supplies, Cat. No. 06471-AB1) and allowed to air dry. Cells were visualized using a Sigma 500 VP FESEM electron microscope. A representative light micrograph of P127-B2a is provided in FIG. 2A and a representative electron micrograph is provided in FIG. 2B. The cells appear as curly rods.

P127-B2a was also assessed for its ability to form spores. Using two distinct sporulation-inducing methods (heat-shock and chemical-shock), P127-B2a was found to be non-sporulating (Table 5). Clostridium butyricum (ATCC 19398) was used as a positive control.

TABLE 4
Assessment of sporulation.

| | Sporulation | Sporulation |
|---|---|---|
| | with heat- | with ethanol- |
| Strain | shock method (%) | shock method (%) |
| C. butyricum ATCC 19398 | 31.75 | 24.29 |
| P127-B2a | 0.0 | 0.0 |

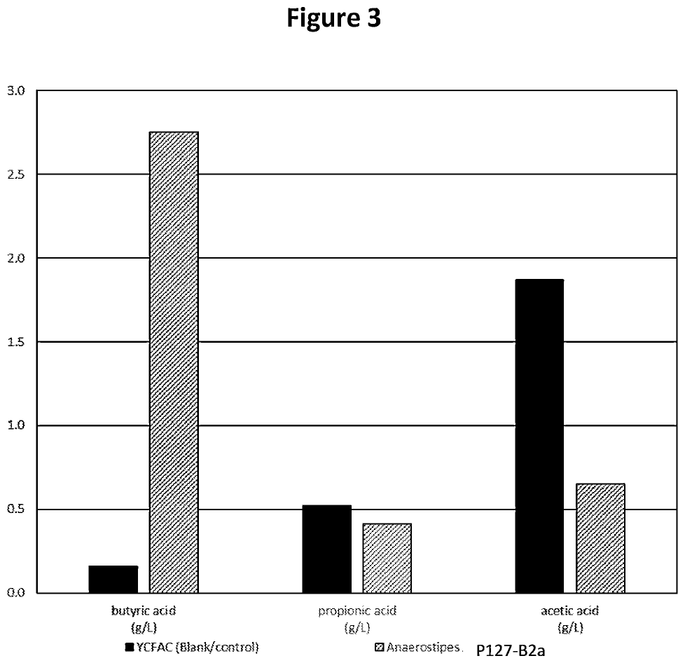

P127-B2a was also assessed for its production of short-chain fatty acids (SCFAs) which are found to contribute to the maintenance of intestinal homeostasis through multiple mechanisms (Lee and Hase, Nat Chem Biol 10(6):416-424 (2014); Hoeppli et al., Front Immunol 6:61 (2015); Koh et al., Cell 165(6):1332-1345 (2016)). SCFAs produced by human gut microbes include propionate, acetate and lactate. The SCFA production profile of P127-B2a was evaluated after 72 hours of growth in batch culture in YCFAC media. Non-inoculated YCFAC media was used as a negative control. As seen in FIG. 3, butyric acid is the most abundant SCFA produced by P127-B2a and there is dramatic utilization of acetic acid by the strain.

### Example 4

### In vitro Functional Activity of Anaerostipes rhamnosivorans P127-B2a

This example describes studies of the activity of Anaerostipes rhamnosivorans P127-B2a in in vitro human epithelial, macrophage, monocyte and dendritic cell models.

### Example 4.1

### Preparation of Freshly Cultured Bacterial Strains for Cell Culture Assays

Freshly cultured bacteria from overnight cultures were prepared in anaerobic conditions. Bacteria were centrifuged at 4300×g for four minutes. Bacteria were washed once with pre-reduced anaerobic PBS (Gibco). Working stock solutions were prepared by resuspending washed bacteria with anaerobic PBS to the total surface area of ˜1×10\hat{ }10 μm². Total surface area was calculated by determining the number of particles (bacterial cells) in solution, then multiplying the total number of particles by the average surface area (μm²) of each particle, as measured by a particle counter (Beckman Coulter Counter). 10-fold serial dilutions were made using anaerobic PBS for specific assays.

### Example 4.2

### HT29-MTX-E12 Human Epithelial Barrier In vitro Assay

Symptoms of increased intestinal permeability can be reversed by reducing the levels of an inflammatory cytokine TNF-α with anti-TNF-α therapy (Michielan et al., Mediators Inflamm. 2015:628157 (2015)). Finding novel therapeutics that can reduce gut permeability and barrier disruption remains an important goal in developing treatments for various disorders. The functional activity of Anaerostipes rhamnosivorans P127-B2a was evaluated on human epithelial cells in the presence of TNF-α, as a model of gut barrier damage and permeability. The confluent HT29-MTX-E12 human epithelial cell line forms a polarized monolayer and produces mucin which are two major characteristics of primary human intestinal epithelial cells (Dolan et al., PLoS One. 7(10): e47300 (2012)).

The HT29-MTX-E12 human epithelial cell line (Sigma Aldrich cat#12040401-1VL) was cultured in 37° C. and 5% CO₂ using high glucose DMEM containing 4.00 mM L-glutamine, 4500 mg/L and sodium pyruvate (HyClone) supplemented with 10% heat-inactivated FBS (Corning), 100 I.U/mL Penicillin, 100m/mL Streptomycin and 0.292 mg/mL L-glutamine (Corning). Passage number was restricted to 6 passages. Apical compartments of HTS 96-well Transwell plates with 0.4 μm microscopically transparent polyester membrane (Corning) were coated with Type 1 Collagen from rat tail (Sigma Aldrich). The HT29-MTX-E12 human cell line was cultured until 70-80% confluent in T-175 tissue culture flasks. Cells were removed with 0.25% Trypsin 2.21 mM EDTA and counted. 30,000 cells were plated onto the apical compartments of pre-coated transwell plates with DMEM culture medium as above and basal reservoirs of transwell plates were filled with the same medium. Cells were cultured in 37° C. and 5% CO₂ for 18 days until a confluent monolayer was formed as described previously (Hall et al., Journal of Pediatric Surgery 48:353-358 (2013)). Media in the apical compartment and basal reservoir were replaced with new media every two days. One day before the experiment, apical compartment inserts containing the confluent monolayer of HT29-MTX-E12 were transferred to HTS Transwell-96 well receiver plates (Corning). Cells were washed and resuspended with DMEM culture media without antibiotics +non-heat-inactivated FBS (Tissue Culture Biologicals) and basal compartments of the transwell receiver plates were filled with the same.

A working solution of the indicated individual freshly cultured bacteria (Anaerostipes rhamnosivorans P127-B2a or negative control strain at 1×10\hat{ }9 μm²/ml and 1×10\hat{ }8 μm²/ml) or anaerobic PBS control at 10% v/v was added onto the apical compartment of the transwell plates containing confluent HT29-MTX-E12 monolayers. Test articles were centrifuged down to the monolayer at 515×g for four minutes. HT29-MTX-E12 monolayers were co-incubated with test articles for one hour in 37° C. and 5% CO₂. 100 ng/ml TNF-α (InvivoGen) was added to the basal compartment of the transwell plate for specific experiments. Trans-epithelial electrical resistance (TEER) across the cell barrier was measured at 0 (before addition of bacteria) and 24 hours after addition of TNF-α using the STX100C electrode attached to EVOM2 Volt/Ohm (TEER) Meter (World Precision Instruments). Cells were washed with HBSS (Corning) once and culture media in the apical and basal compartments was replaced by HBSS before TEER at the 24-hour time point was measured.

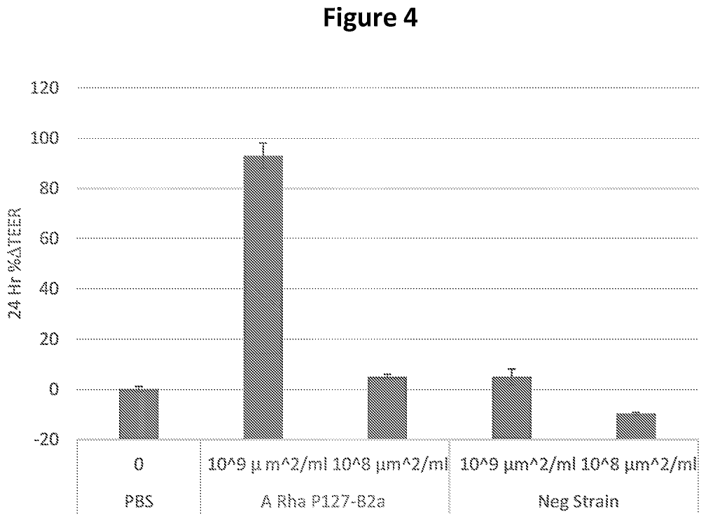

FIG. 4 shows a significant increase in TEER (measurement of cell monolayer integrity) when 1×10\hat{ }9 μ²/ml of Anaerostipes rhamnosivorans P27-B2a was incubated with the HT29-MTX-E12 monolayer in the presence of TNF-α compared to the PBS control and negative control strain. This indicates that Anaerostipes rhamnosivorans can protect human epithelial cells from cytokine-induced barrier damage and can reduce intestinal barrier permeability.

### Example 4.3

### Human Macrophage and Monocyte In vitro Cytokine and Chemokine Assay

The THP-1 human monocyte cell line (ATCC cat# TIB-202) was cultured in 37 ° C. and 5% CO₂ using RPMI 1640 containing 2.05mM L-glutamine (Corning) supplemented with 10% heat-inactivated FBS (Corning), 100 I.U/mL Penicillin, 100m/mL Streptomycin and 0.292 mg/mL L-glutamine (Corning). Passage number was restricted to 8 passages. The THP-1 human monocyte cell line was grown until 70-80% confluent. Cells were counted and resuspended in culture media. 100,000 cells were plated per well onto 96 well plates. THP-1 human macrophages were made by culturing the THP-1 human monocyte cells with 10 ng/mL phorbol 12-myristate 13-acetate (PMA) (InvivoGen) for 24 hours followed by 20ng/mL IL-4 (R&D Systems) and 20 ng/mL IL-13 (R&D Systems) for 48 hours in 37° C. and 5% CO₂ as described previously previously (Genin et al., BMC Cancer 15:577 (2015)). One day before the experiment, cells were washed and resuspended in RPMI culture media without antibiotics containing 20 ng/mL IL-4 and 20 ng/mL IL-13.

A working stock solution of the indicated individual freshly cultured, anaerobic PBS or 500 ng/ml LPS was added onto THP-1 macrophages at 10% v/v and centrifuged down onto the THP-1 cells at 515×g for four minutes. The test articles, controls and THP-1 macrophages were co-incubated for 3 hours in 37° C. and 5% CO₂. The coculture media was replaced by fresh RPMI culture media supplemented with antibiotics to limit excess bacteria growth. THP-1 cells were incubated after culture media replacement for 15 hours in 37° C. and 5% CO₂. THP-1 cell supernatants were collected and analyzed by ELISA. Levels of CCL-18, IL-10, TNFa, IL-1β, and IL12-p40 in culture supernatants were quantified by using commercial enzyme-linked immunosorbent assay (ELISA) kits from Biolegend or R&D

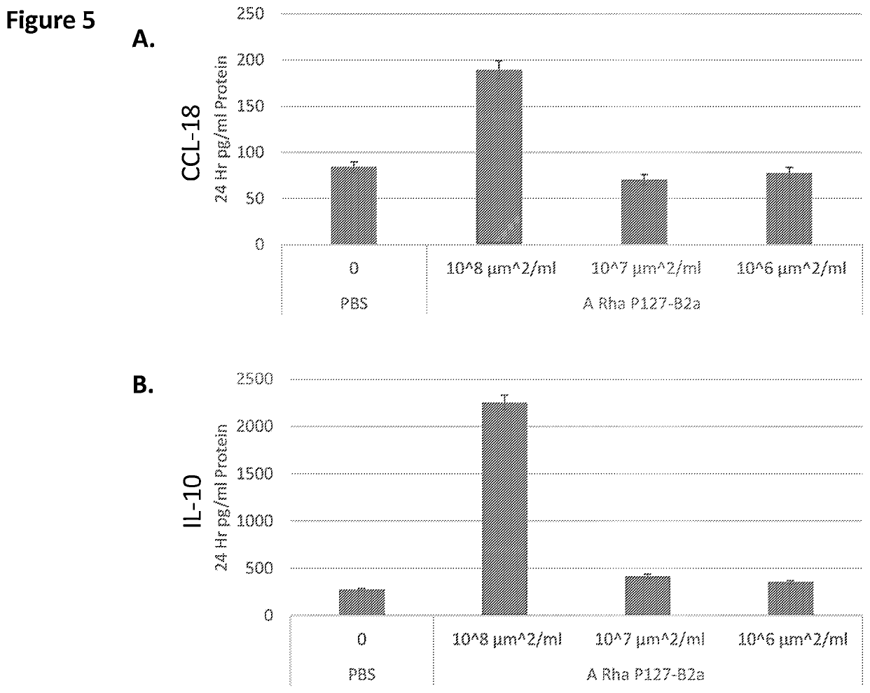

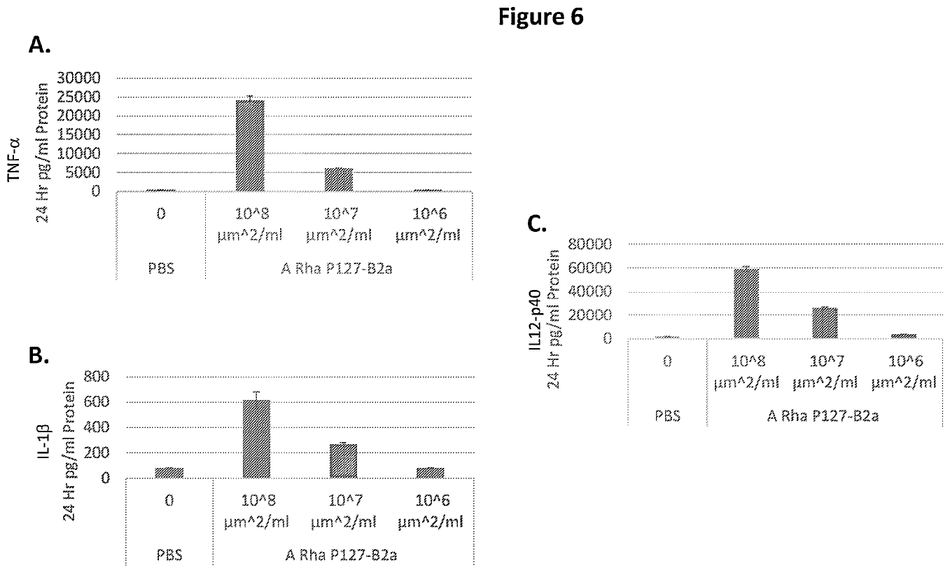

Systems with TMB detection according to manufacturer's specifications. FIG. 5 shows a significant, dose-dependent increase in the production of the anti-inflammatory cytokines (A) CCL-18 and (B) IL-10 by THP-1 macrophages induced by Anaerostipes rhamnosivorans P127-B2a. FIG. 6 shows similar significant, dose-dependent increases in the production of (A) TNFα, (B) IL-1β, and (C) IL12-p40 in THP-1 macrophages induced by Anaerostipes rhamnosivorans P127-B2a.

### Example 4.4

### Human PBMC In vitro Cytokine Assay

Trima residual blood product containing concentrated blood mononuclear cells was obtained from anonymous donors through Blood Centers of the Pacific (San Francisco, CA) and processed within 24 hours of collection. Blood samples were tested negative for HIV, HBV, HCV, HTLV, Syphilis, West Nile Virus and Zika Virus. PBMC were isolated using a ficoll gradient as described previously (Sim et al., J. Vis. Exp. (112), e54128 (2016)). Briefly, 50 mL of Trima residual was diluted with 50 mL of sterile PBS (Gibco) and 25 mL was overlaid on 15 mL Ficoll-Paque Plus (GE Healthcare) in 50 mL conical tubes. The samples were centrifuged at 450×g for 30 min at room temperature and allowed to stop without brake. The PBMC interphase was collected, washed with PBS and resuspended in RPMI 1640 containing 2.05 mM L-glutamine (Corning) supplemented with 10% heat-inactivated FBS (Tissue Culture Biologicals) and 0.292 mg/mL L-glutamine (Corning). The cells were maintained by incubation in 37° C. and 5% CO₂ and used for assay evaluation within 24 h or frozen for later use. Cells were cryopreserved in RPMI 1640 supplemented with 50% FBS and 10% DMSO (Sigma Aldrich) at a concentration of 5×10⁷ cells/mL and stored in liquid nitrogen until ready for use.

Human PBMCs, used immediately after isolation or thawed from cryo-storage, were diluted to 5×10⁶ cells/mL in RPMI 1640 containing L-glutamine (Corning) supplemented with 10% heat-inactivated FBS (Tissue Culture Biologicals) and 0.292 mg/mL L-glutamine (Corning). A 100 μL aliquot of the 5×10⁶ cells/mL cell suspension was added to each well within a round-bottom 96 well plate and cultured for 24 hours at 37° C. and 5% CO₂ before addition of test articles.

Test articles were prepared and added as described for other assays. After 3 hours of incubation in 37° C. with 5% CO_{2,} the plates containing cocultures were centrifuged (515 x g; four minutes), media removed, and replaced with RPMI 1640 supplemented with 10% heat-inactivated FBS, L-glutamine, and penicillin/streptomycin antibiotic. Culture plates were then incubated for an additional 15 h at 37° C. and 5% CO₂. The plates were centrifuged (515×g; four minutes) and supernatant was collected and analyzed by a custom U-plex multiplex kit from Meso Scale Discovery according to manufacturer's instructions. Results were averaged from 4 human donors with two experimental replicates from each donor.

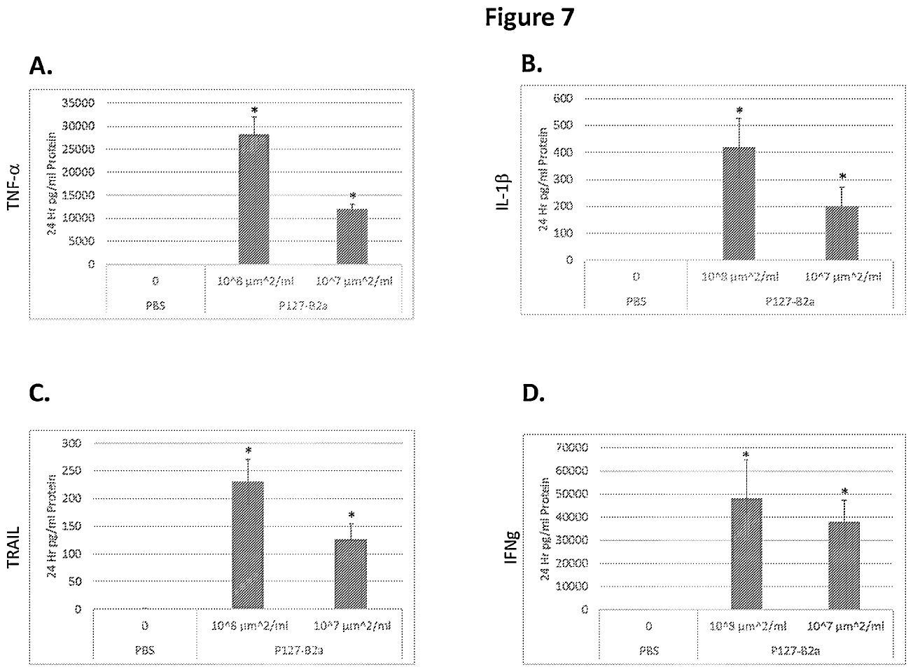

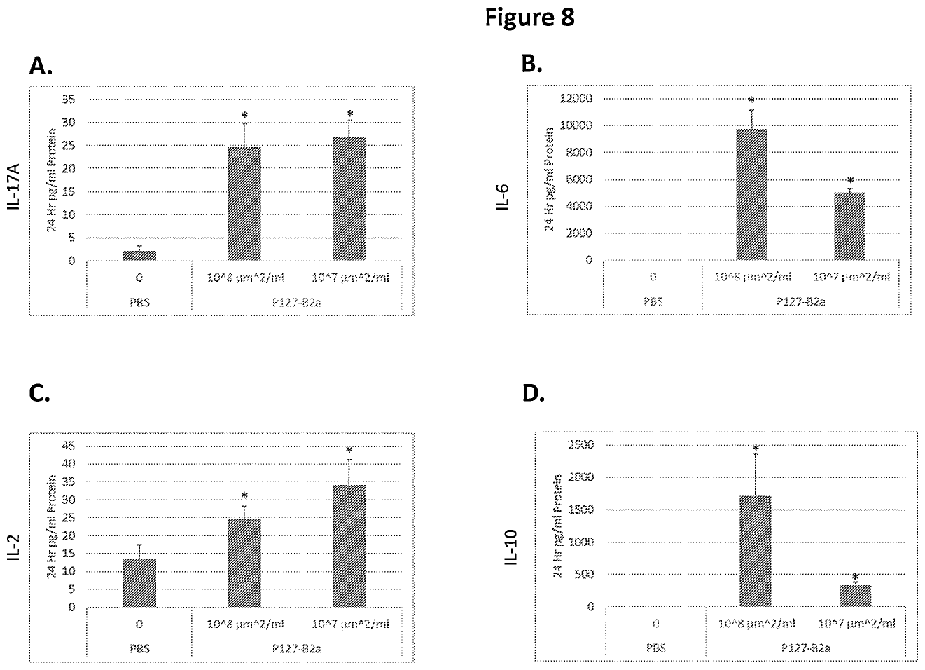

FIG. 7 shows a significant, dose-dependent increases in the production of: (A) TNFa, (B) IL-1β, (C) TRAIL, and (D) IFNy by human PBMCs induced by Anaerostipes rhamnosivorans P127-B2a. FIG. 8 shows similar significant, dose-dependent increases in the production of: (A) IL-17A, (B) IL-6, (C) IL-2, and (D) IL-10 in human PBMCs induced by Anaerostipes rhamnosivorans P127-B2a.

### Example 4.5

### Human Monocyte-derived Dendritic Cell In vitro Cytokine Assay

Cryopreserved PBMCs were thawed in a 37° C. water bath, diluted in warm RPMI 1640 supplemented with 10% heat-inactivated FBS and L-glutamine, and centrifuged (515×g; four minutes). Cells were resuspended in PBS buffer containing 0.5% bovine serum albumin (BSA) and 2 mM EDTA and CD14+monocyte cells were isolated by selection using Miltenyi CD14 Microbeads according to manufacturer's directions. Isolated CD14+ monocytes were cultured in RPMI 1640 supplemented with 10% heat-inactivated FBS, L-glutamine, penicillin/streptomycin antibiotic, 50 ng/mL recombinant human IL-4 (R&D

Systems), and 100 ng/mL recombinant human GM-CSF (Biolegend). Media was replenished on days 3 and 6. On day 7 after isolation, cells were diluted to 5×10⁵ cells/mL in RPMI 1640 containing L-glutamine (Corning) supplemented with 10% heat-inactivated FBS (Tissue Culture Biologicals) and 0.292 mg/mL L-glutamine (Corning). A 100 μl aliquot of the 5×10⁵ cells/mL cell suspension was added to each well within a flat-well 96 well plate and cultured for 24 hours at 37° C. and 5% CO₂ before addition of test articles.

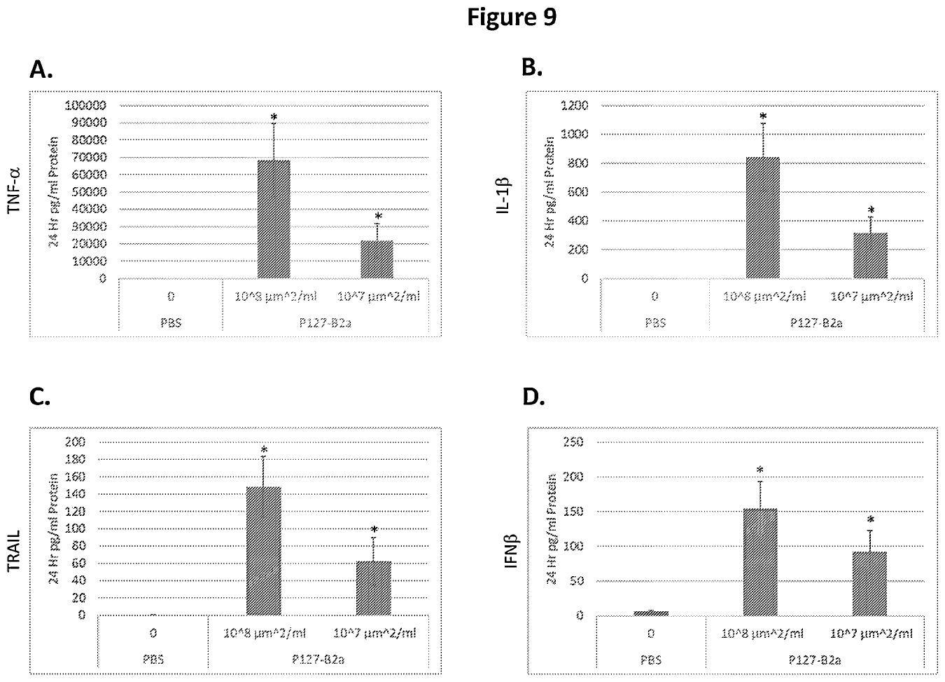

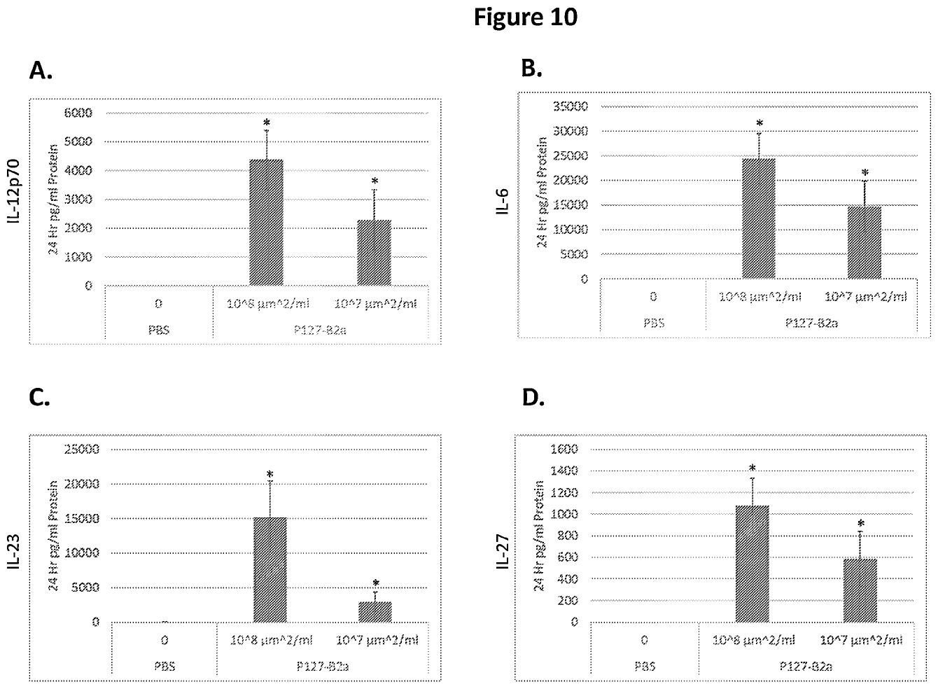

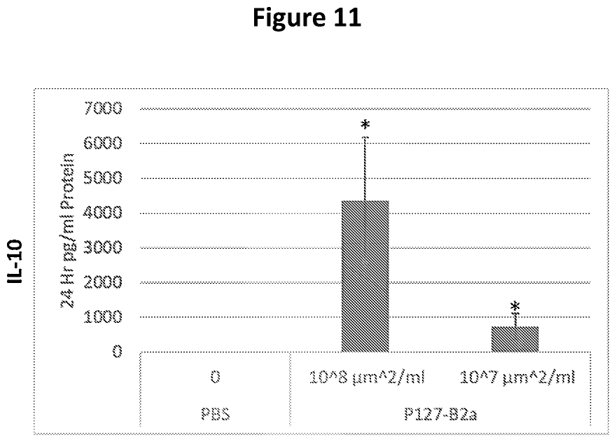

Test articles were prepared and added as described for other assays. After 3 hours of incubation in 37° C. with 5% CO_{2,} the plates containing cocultures were centrifuged (515 x g; four minutes), media removed, and replaced with RPMI 1640 supplemented with 10% heat-inactivated FBS, L-glutamine, and penicillin/streptomycin antibiotic. Culture plates were then incubated for an additional 15 h at 37° C. and 5% CO₂. The plates were centrifuged (515×g; four minutes) and supernatant was collected and analyzed by a custom U-plex multiplex kit from Meso Scale Discovery according to manufacturer's instructions. Results were averaged from 4 human donors with two experimental replicates from each donor. FIG. 9 shows a significant, dose-dependent increases in the production of: (A) TNFa, (B) IL-1β, (C) TRAIL, and (D) IFNβ by human monocyte-derived dendritic cells (moDCs) induced by Anaerostipes rhamnosivorans P127-B2a. FIG. 10 shows similar significant, dose-dependent increases in the production of: (A) IL-12p70, (B) IL-6, (C) IL-23, and (D) IL-27 in human moDCs induced by Anaerostipes rhamnosivorans P127-B2a. FIG. 11 shows a dose dependent increase in the production of IL-10 by moDCs induced by Anaerostipes rhamnosivorans P127-B2a.

### Example 5

### In vivo Functional Activity of Anaerostipes rhamnosivorans P127-B2a

Anaerostipes rhamnosivorans P127-B2a was tested for efficacy in two different well-validated mouse models of inflammatory skin disease: (1) imiquimod (IMQ)-induced psoriasis; and (2) oxazolone-induced atopic dermatitis.

5.1 Imiquimod (IMQ)-Induced Psoriasis

Psoriasis is an immune-mediated chronic inflammatory skin disorder characterized by scaly, reddened skin lesions and thickening of the affected skin as well as epidermal and/or dermal cellular and histo-pathological changes. Topical application of Toll-like Receptor 7/8 activator, imiquimod (IMQ) is known to cause psoriasis-like skin inflammation both in humans and mice. See, e.g. van der Fits L. et al. Imiquimod-induced psoriasis-like skin inflammation in mice is mediated via IL23/IL17 axis; J Immunology, 2009, 182:5836-5845.

In this study, BALB/c mice received daily topical application of 5% IMQ cream (47 mg/day) on the back skin (area: 4 cm x 2 cm) for 6 consecutive days. Test items were administered once daily from Days -7 to termination and included live purified individual bacterial strains or vehicle (bacteria freezing media) by oral gavage once daily, approximately 1 to 2 hours after IMQ application. Animals receiving positive control were given 0.05% clobetasol cream (62.5 mg/day) applied topically approximately 1 hour after IMQ application. Skin evaluations were performed daily starting at Day 2, including evaluation of back skin thickness using an Engineering micrometer.

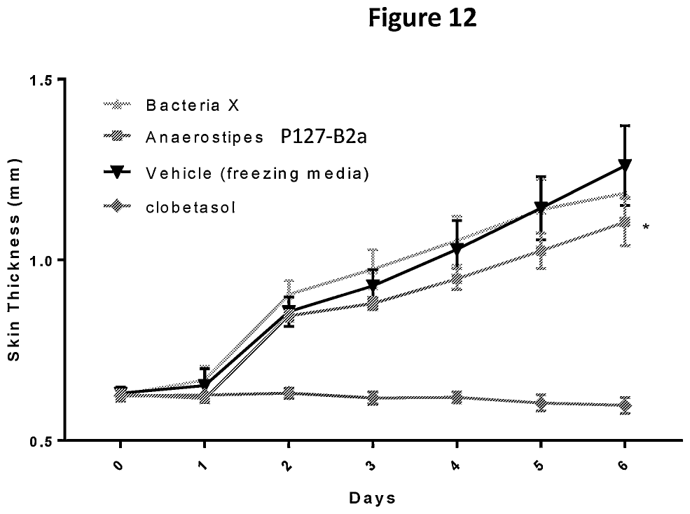

As shown in FIG. 12, administration of Anaerostipes rhamnosivorans P127-B2a as well as clobetasol led to a reduction in imiquimod-induced skin thickness compared to the vehicle control (at day 6), whereas administration of bacterial strain X did not.

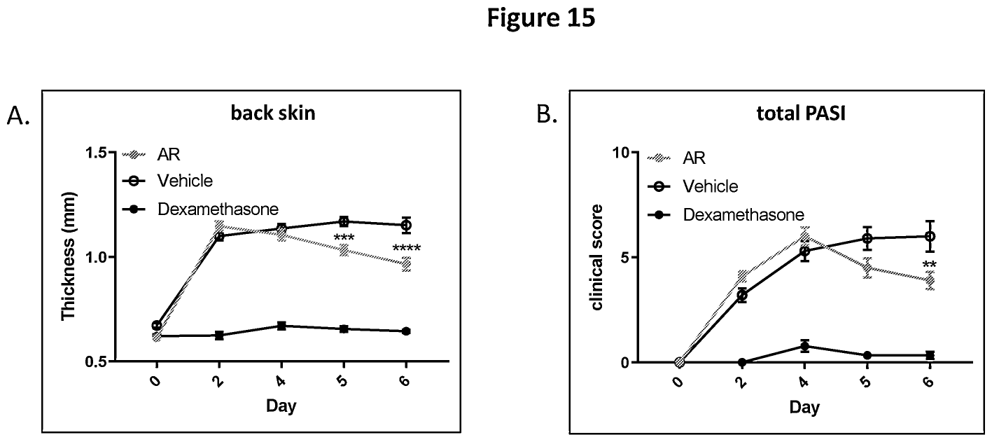

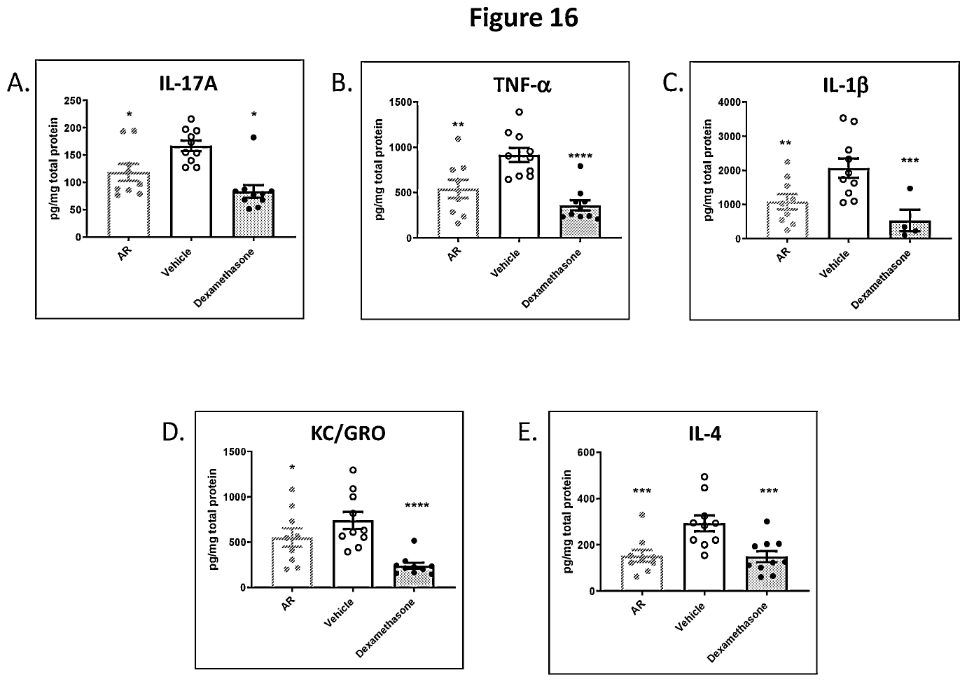

In a second similar study, administration of Anaerostipes rhamnosivorans P127-B2a (AR) as well as dexamethasone (positive control) led to a significant reduction in imiquimod-induced skin thickness (FIG. 15A) and total psoriasis area and severity index (PAST) score based on erythema (0-4), edema (0-4) and desquamation (0-4) (FIG. 15B) compared to the vehicle control (at day 6). Skin samples were harvested at termination and assessed by Meso Scale Discovery (MSD) analysis for levels of pro-inflammatory cytokines and chemokines (normalized to homogenate protein concentration). As shown in FIG. 16, oral dosing of Anaerostipes rhamnosivorans P127-B2a (AR) led to significantly reduced skin levels of (A) IL-17A, (B) TNF-α, (C) IL-1β, (D) KC/GRO (keratinocyte chemoattractant (KC) chemokines CXCL1/2, mouse homologues of human growth-regulated oncogenes (GRO)), and (E) IL-4, in comparison to their skin levels in vehicle control-treated mice.

5.2 Atopic Dermatitis Models

Atopic dermatitis, also known as atopic eczema, is a type of inflammation that results in itchy, red, swollen and cracked skin which thickens over time. Induction of atopic dermatitis in mice through the topical application of oxazolone in mice has been previously reported. See e.g. Hatano et al., 2009 Maintenance of an acidic stratum corneum prevents emergence of murine atopic dermatitis. J Invest Dermat 129: 1824-1835; and Ishii et al., 2013 Antipruritic effect of the topical phosphodiesterase 4 inhibitor E6005 ameliorates skin lesions in a mouse atopic dermatitis model. J Pharmacol Exp Ther 346:105-112.

In a first atopic dermatitis study, on Day 0, BALB/c mice were sensitized with a single topical application of 60 μL 0.3% oxazolone (Ox) on the back skin. Starting from Day 5, the animals were topically given Ox challenge (60 0.3%) on the back once every two days until termination. Test items were administered once daily from Days -7 to termination and included live purified individual bacterial strains or vehicle (bacteria freezing media) by oral gavage once daily. Animals receiving positive control were given topical application of 0.05% clobetasol cream (62.5 mg/day) on the back from Days 1 to 21. On days of challenges, test items and clobetasol were administered 1 to 2 hours after oxazolone application. Skin evaluations were performed every other day starting at Day 5, including evaluation of affected skin for erythema or redness, and back skin scaling, in accordance with the following scales:

Skin erythema or redness:

| | |
|---|---|
| None | =0 |
| Slightly Red | =1 |
| Moderately Red | =2 |
| Markedly Red | =3 |
| Very Markedly Red | =4 |

Skin Scales:

| | |
|---|---|
| None | =0 |
| Slightly Scaly | =1 |
| Moderately Scaly | =2 |
| Markedly Scaly | =3 |
| Very Markedly Scaly | =4 |

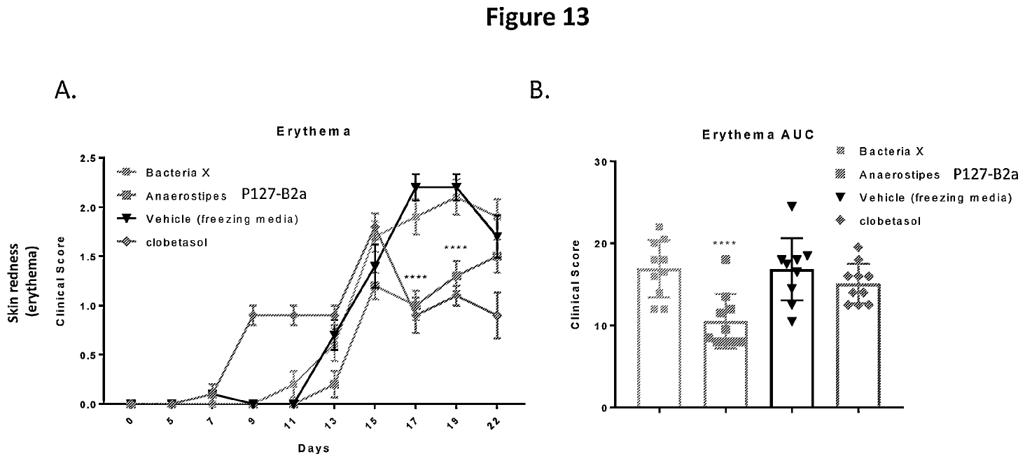

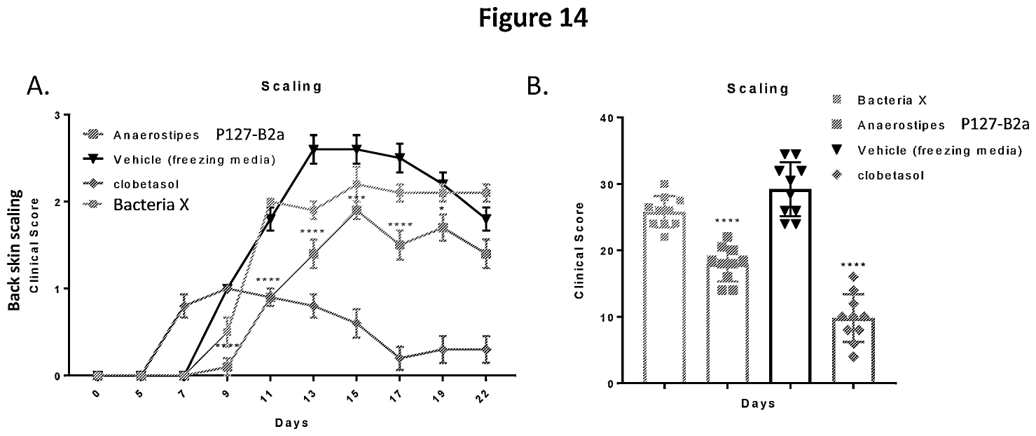

As shown in FIG. 13, administration of Anaerostipes rhamnosivorans P127-B2a as well as clobetasol led to a reduction in oxazolone-induced skin redness compared to the vehicle control, whereas administration of bacterial strain X did not. Similarly, as shown in FIG. 14, administration of Anaerostipes rhamnosivorans P127-B2a as well as clobetasol led to a reduction in oxazolone-induced back skin scaling compared to the vehicle control), whereas administration of bacterial strain X did not.

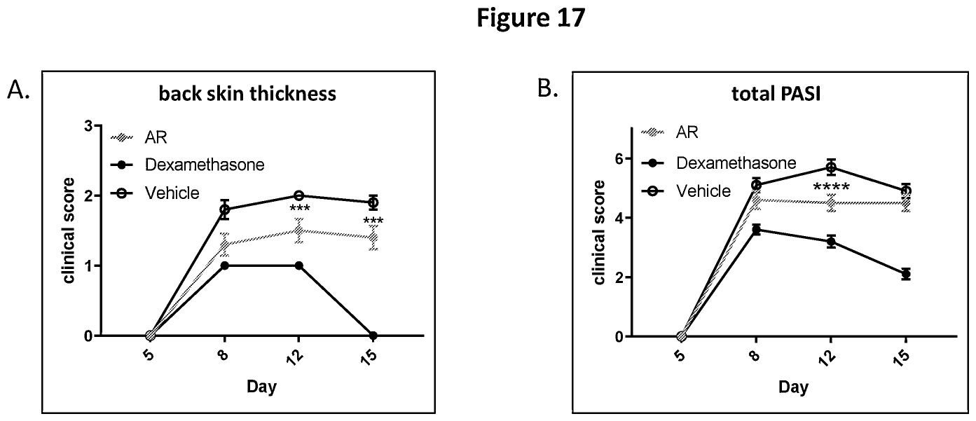

A second atopic dermatitis study was conducted in which 0.15% 2, 4-dinitrofluorobenzene (DNFB) was topically applied on days 1, 5, 8, 12 and 15 of a 15-day study to induce atopic dermatitis-like lesions in mice. Test items were administered once daily from Days 0 to 15 and included live purified individual bacterial strains or vehicle (bacteria freezing media) by oral gavage. Animals receiving positive control were given oral administration of Dexamethasone (10 mL/kg) from Days 0 to 15. On Days 5-8-12-15, skin clinical scores were recorded based on erythema (0-4), edema (0-4) and desquamation (0-4). As shown in FIG. 17, administration of Anaerostipes rhamnosivorans P127-B2a (AR) as well as dexamethasone (positive control) led to a significant reduction in DNFB-induced skin thickness at days 12-15 (FIG. 17A) and total skin clinical score at day 12 (FIG. 17B) compared to the vehicle control.

5.3 Anaerostipes rhamnosivorans P127-B2a in combination with additional strains

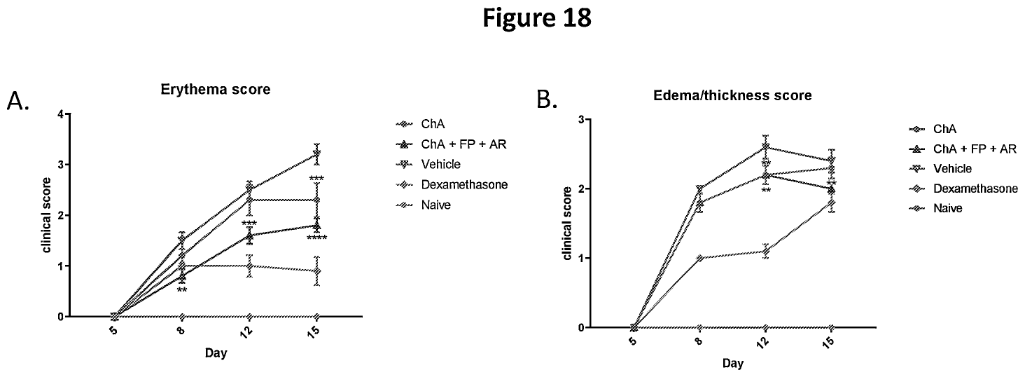

In an additional DNFB-induced atopic dermatitis study, skin conditions were assessed in animals administered the combination of Anaerostipes rhamnosivorans P127-B2a (AR) plus a Christensenella strain, Christensenella sp. P152-H6d (ChA), plus a Faecalibacterium prausnitzii strain, Faecalibacterium prausnitzii P162-C10a (FP), in equal amounts (once daily) for the duration of the study. Single strain test groups were administered Christensenella sp. P152-H6d alone, once daily for the duration of the study. The negative control group received vehicle only once daily for the duration of the study, while the positive control group received Dexamethasone. As shown in FIG. 18, administration of ChA alone resulted in a reduction of erythema (redness) (FIG. 18A) as well as edema (thickness) (FIG. 18B) compared to administration of vehicle only. However, administration of the triple combination of ChA +FP +AR resulted in an enhanced reduction of both erythema and edema compared to administration of ChA only.

### INCORPORATION BY REFERENCE

The entire disclosure of each of the patent and scientific documents referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting on the disclosure described herein.

Scope of the disclosure is thus indicated by the appended claims rather than by the foregoing description, and all changes that come within the meaning and range of equivalency of the claims are intended to be embraced therein.

We claim:

1. A composition comprising:
a bacterial strain of the genus Anaerostipes that comprises a 16s rRNA gene sequence with at least about 98% sequence identity to the polynucleotide sequence of SEQ ID NO: 1; and
an excipient, diluent, and/or carrier;
wherein the bacterial strain is lyophilized, freeze dried, or spray dried.

2. The composition of claim 1, wherein the bacterial strain is capable of increasing production of IL-10 by a human macrophage, monocyte, peripheral blood mononuclear cell or monocyte-derived dendritic cell in vitro.

3-5. (canceled)

6. The composition of claim 1, wherein the bacterial strain comprises a nucleotide sequence having at least about 70% identity to any one of SEQ ID NOs: 2-52.

7. The composition of claim 1, wherein the bacterial strain comprises a genome having at least 95% average nucleotide identity (ANI) with the genome of Anaerostipes sp. strain P127-B2a, deposited under accession number DSM 33275.

8. (canceled)

9. The composition of claim 1, wherein the bacterial strain is Anaerostipes sp. P127-B2a, deposited under accession number DSM 33275.

10. The composition of claim 1, wherein the composition is formulated as an enteric formulation.

11. The composition of claim 10, wherein the enteric formulation is formulated as a capsule, tablet, caplet, pill, troche, lozenge, powder, or granule.

12. The composition of claim 1, wherein the composition is formulated as a suppository, suspension, emulsion, or gel.

13. The composition of claim 1, wherein the composition comprises at least 1×10³ CFU of the bacterial strain.

14. The composition of claim 1, wherein the composition comprises a therapeutically effective amount of the bacterial strain sufficient to prevent or treat a disorder is administered to a subject in need thereof.

15-17. (canceled)

18. The composition of claim 1, wherein the excipient is selected from the group consisting of cellulose, polyvinyl pyrrolidone, silicon dioxide, stearyl fumarate or a pharmaceutically acceptable salt thereof, and any combination(s) thereof

19. The composition of claim 1, wherein the composition further comprises a cryoprotectant.

20. The composition of claim 19, wherein the cryoprotectant is selected from the group consisting of a fructooligosaccharide, trehalose, and a combination thereof

21. (canceled)

22. The composition of claim 1, wherein the composition is suitable for bolus administration or bolus release.

23-26. (canceled)

27. The composition of claim 1, wherein upon storage for 6 months at 4° C., the composition loses at most 3 log colony forming units (cfus) of the bacterial strain.

28. A food product comprising the composition of a claim 1.

29. A method of treating a dysbiosis, gastrointestinal disorder, inflammatory disorder, or skin disorder in a subject in need thereof, the method comprising administering a therapeutically effective amount of the composition of claim 1 to the subject.

30. A method of modifying a gut microbiome in a subject, the method comprising administering a therapeutically effective amount of the composition of claim 1 to the subject.

31-35. (canceled)

36. A method of treating non-alcoholic fatty liver disease (NAFLD), non-alcoholic steatohepatitis (NASH), metabolic syndrome, insulin deficiency, insulin resistance-related disorders, insulin sensitivity, glucose intolerance, pre-diabetes, diabetes, high body mass index (BMI), excess adiposity, obesity, excess weight, cardiovascular disease, atherosclerosis, hyperlipidemia, hyperglycemia, abnormal lipid metabolism, or hypertension in a subject in need thereof, the method comprising administering a therapeutically effective amount of the composition of claim 1 to the subject.

37. The method of claim 29, further comprising administering a prebiotic to the subject.

38. (canceled)
